(19) United States
(12) Patent Application Publication
(10) Pub. No.: US 2025/0171450 A1
(43) Pub. Date: May 29, 2025

(54) NOVEL COELENTERAZINE DERIVATIVES

(71) Applicant: National Institute of Advanced Industrial Science and Technology, Chiyoda-ku, Tokyo (JP)

(72) Inventors: Shojiro MAKI, Chofu-shi, Tokyo (JP); Nobuo KITADA, Chofu-shi, Tokyo (JP); Sung-Bae KIM, Chiyoda-ku, Tokyo (JP)

(73) Assignee: National Institute of Advanced Industrial Science and Technology, Chiyoda-ku, Tokyo (JP)

(21) Appl. No.: 18/841,467

(22) PCT Filed: Feb. 21, 2023
(86) PCT No.: PCT/JP2023/006307

§ 371 Date: Aug. 26, 2024

(30) Foreign Application Priority Data
Feb. 28, 2022 (JP) 2022-030488

(51) Int. Cl. C07D 487/04

(52) U.S. Cl. CPC C07D 487/04

(57) ABSTRACT

The present disclosure is directed to providing novel coelenterazine derivatives that exhibit high luminescence brightness and have enzyme specificity, and the solution is a coelenterazine derivative represented by the following general formula (1) or (2):

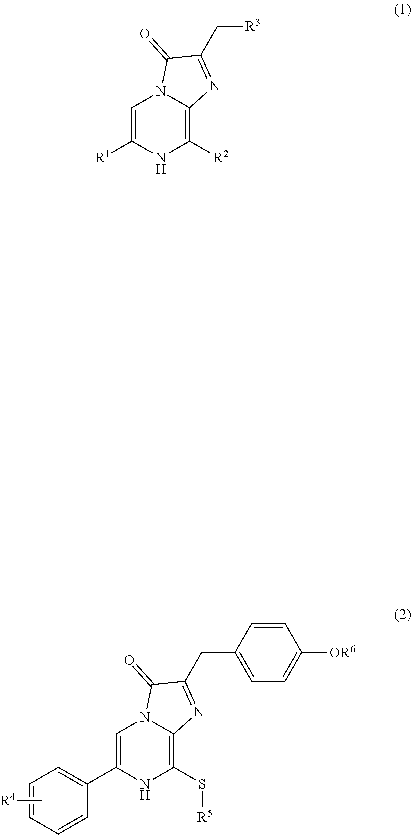

in the general formula (1), R¹ has a certain bicyclic structure, R² is represented by —R^{2′} or —CH₂—R^{2′} where R^{2′} has a certain ring structure, and R³ has a certain ring structure; and in the general formula (2), R⁴ is represented by —(CH₂)ₙ—OR⁴⁻¹, —N(R⁴⁻¹)₂, or —CF₃ where each R⁴⁻¹ is independently hydrogen or a hydrocarbon group with 1 to 3 carbon atoms, n is an integer from 0 to 3, R⁵ has a certain ring structure, and R⁶ is hydrogen or a hydrocarbon group with 1 to 3 carbon atoms.

### TECHNICAL FIELD

The present disclosure is regarding to novel coelenterazine derivatives.

### BACKGROUND

Technologies for visualizing molecular events in living organisms are essential for diagnosing various diseases and developing their treatment methods. Bioluminescence-based techniques are the mainstream in the present visualization technologies, and the technologies utilizing the luminescence systems of fireflies and marine organisms have been developed to date. Among these, the luminescence systems of marine organisms have attracted great attention due to their high luminescence brightness, small molecular weight of the luminescent enzymes, the simplicity of the luminescence system, etc. Such systems have been favorably used even for imaging viruses in recent years. As an example of the marine organism-derived luminescence systems, it is known that natural coelenterazine (nCTZ), which is a luminescent substrate, emits light with a wavelength of about 480 nm through its oxidation reaction catalyzed by the luminescent enzyme of marine organisms.

Meanwhile, to observe molecular events occurring deep tissues within living organisms, there have been great demands on such luminescence systems that luminesce in much longer wavelength regions and have higher brightness, compared to conventional systems. Research has directed toward utilizing bioluminescent substrates as labeling materials to visualize lesions deep within the living organism. For example, luminescence systems that work in longer wavelength regions and have higher brightness are required for observing deep tissues within the living subjects, and coelenterazine derivatives with modified luminescence properties through structural modifications have been developed (PTL 1 and NPLs 1-3).

### CITATION LIST

### Patent Literature

PTL 1: JP 2018-165265 A

### Non-Patent Literature

NPL 1: Jiang, T.; Du, L.; Li, M. Photochem. Photobiol. Sci. 2016, 15 (4), 466-480.

NPL 2: Kaskova, Z. M.; Tsarkova, A. S.; Yampolsky, I. V. . Soc. Rev. 2016, 45 (21), 6048-6077.

NPL 3: Nishihara, R.; Suzuki, H.; Hoshino, E.; Suganuma, S.; Sato, M.; Saitoh, T.; Nishiyama, S.; Iwasawa, N.; Citterio, D.; Suzuki, K. Chem. Commun. (Camb). 2015, 51 (2), 391-394.

### SUMMARY

### Technical Problem

However, it is known that modifying the structure of natural coelenterazine (nCTZ), which is a luminescent substrate, significantly decreases the luminescence brightness. Therefore, it is highly necessary to modify the structure of luminescent substrates while maintaining the luminescence brightness.

Additionally, it is challenging to modulate the luminescence brightness, luminescence wavelength, and luminescent enzyme specificity of conventional marine organism-derived luminescence systems. If it becomes possible to modulate the luminescence brightness, luminescence wavelength, and enzyme specificity of luminescence systems, it enables simultaneous measurement feature, high-sensitivity, and rapid visualization of multiple in vivo phenomena such as molecular events and cancer metastasis. For example, exerting a specific luminescent enzyme to luminesce among many coexisting luminescent enzymes can be achieved by adding a unique luminescent substrate that luminesce only for a specific enzyme.

However, modulating the luminescence brightness and luminescent enzyme specificity of marine organism-derived luminescence systems is highly challenging.

Therefore, the present disclosure is directed to providing novel coelenterazine derivatives that exhibit high luminescence brightness and enzyme specificity for addressing the above-mentioned conventional technical limitations.

Furthermore, the present disclosure is also directed to providing novel coelenterazine derivatives that exhibit luminescence in the longer wavelength region, besides high luminescence brightness and high enzyme specificity.

### Solution to Problem

We have conducted extensive research to address the above limitations and found that coelenterazine derivatives with specific bicyclic structures and/or specific thioether structures exhibit high luminescence brightness and enzyme specificity, leading to the completion of the present disclosure.

Specifically, the coelenterazine derivative of the present disclosure is a coelenterazine derivative represented by the following general formula (1):

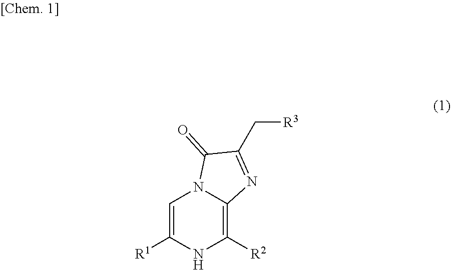

in the general formula (1), R¹ is represented by the following general formula (1-1-1), (1-1-2), (1-1-3), or (1-1-4):

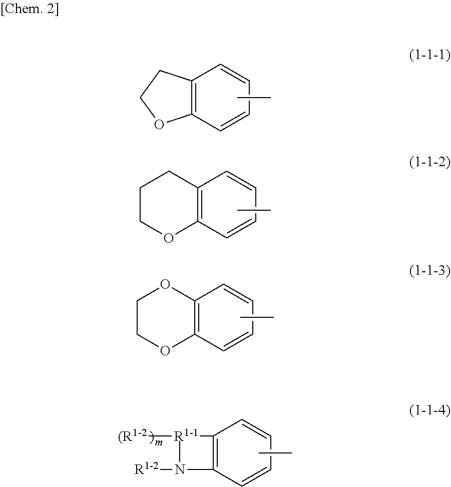

where R¹⁻¹ is a hydrocarbon group with 1 to 4 carbon atoms, each R¹⁻² is independently hydrogen or a hydrocarbon group with 1 to 3 carbon atoms, and m is an integer from 2 to 8,

R² is represented by —R^{2′} or —CH₂—R^{2′}, where R^{2′} is represented by the following general formula (1-2-1), (1-2-2), (1-2-3), (1-2-4), or (1-2-5):

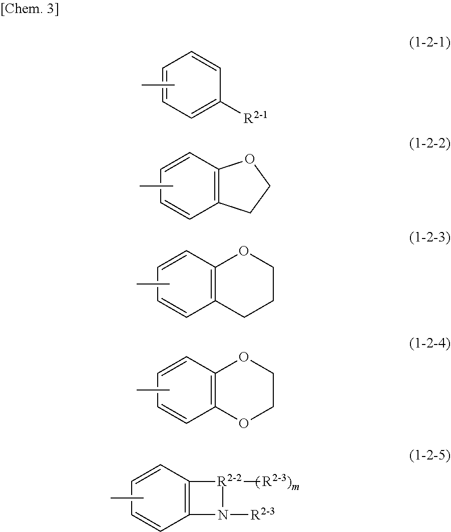

where R²⁻¹ is hydrogen, halogen, —N(R²⁻¹⁻¹)₂, or —OR²⁻¹⁻¹, where each R²⁻¹⁻¹ is independently hydrogen or a hydrocarbon group with 1 to 3 carbon atoms, R²⁻² is a hydrocarbon group with 1 to 4 carbon atoms, each R²⁻³ is independently hydrogen or a hydrocarbon group with 1 to 3 carbon atoms, and m is an integer from 2 to 8, and

R³ is represented by the following general formula (1-3-1), (1-3-2), or (1-3-3):

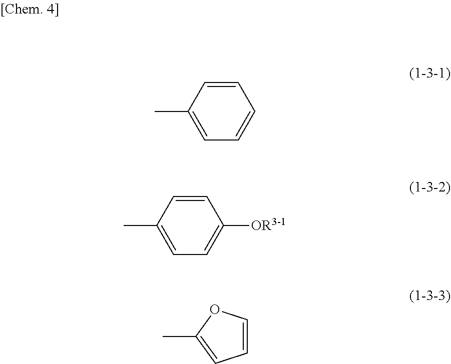

where R³⁻¹ is hydrogen or a hydrocarbon group with 1 to 3 carbon atoms; or the following general formula (2):

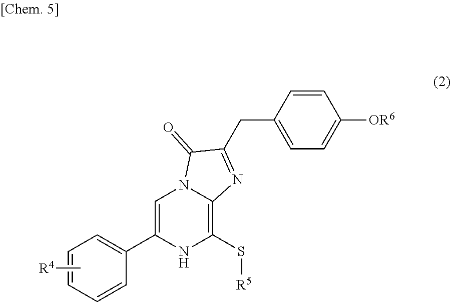

in the general formula (2), R⁴ is hydrogen, —(CH₂)ₙ—OR⁴⁻¹, —N(R⁴⁻¹)₂, or —CF₃, where each R⁴⁻¹ is independently hydrogen or a hydrocarbon group with 1 to 3 carbon atoms, and n is an integer from 0 to 3,

R⁵ is represented by the following general formula (2-5-1), (2-5-2), (2-5-3), (2-5-4), or (2-5-5):

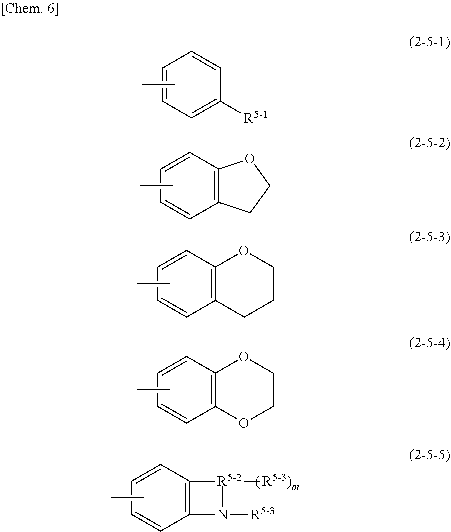

where R⁵⁻¹ is hydrogen, halogen, —N(R⁵⁻¹⁻¹)₂, or —OH, where each R⁵⁻¹⁻¹ is independently hydrogen or a hydrocarbon group with 1 to 3 carbon atoms, R⁵⁻² is a hydrocarbon group with 1 to 4 carbon atoms, each R⁵⁻³ is independently hydrogen or a hydrocarbon group with 1 to 3 carbon atoms, and m is an integer from 2 to 8, and

R⁶ is hydrogen or a hydrocarbon group with 1 to 3 carbon atoms. Such a coelenterazine derivative according to the present disclosure exhibits high luminescence brightness and has enzyme specificity.

Preferably, the coelenterazine derivative of the present disclosure is represented by the above general formula (1), where R¹ is represented by the above general formula (1-1-3). In this case, luminescence brightness is improved.

Preferably, the coelenterazine derivative of the present disclosure is represented by the above formula (1), wherein R² is represented by —CH₂—R^{2′}, where R^{2′} is represented by the above general formula (1-2-1). Also, in this case, luminescence brightness is improved.

Preferably, the coelenterazine derivative of the present disclosure is represented by the above general formula (1), where R³ is represented by the above general formula (1-3-2). Also, in this case, luminescence brightness is improved.

Particularly preferably, the coelenterazine derivative of the present disclosure is represented by the following structural formula (1-1):

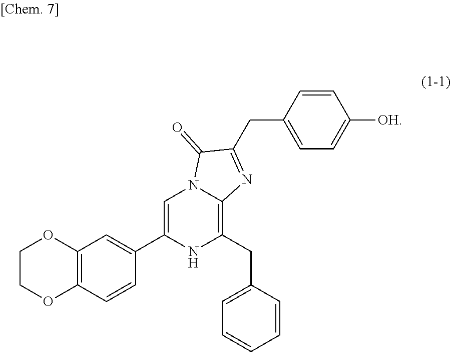 [SMILES: O=c1c(Cc2ccc(O)cc2)nc2c(Cc3ccccc3)[nH]c(-c3ccc4c(c3)OCCO4)cn1-2]

In this case, the luminescence brightness from the luminescent system using the coelenterazine derivative is particularly high.

Preferably, the coelenterazine derivative of the present disclosure is represented by the above general formula (2), where R⁴ is represented by —(CH₂)ₙ—OR⁴⁻¹. In this case, luminescence brightness is improved. Preferably, the coelenterazine derivative of the present disclosure is

represented by the above general formula (2), where R⁵ is represented by the above general formula (2-5-1). Also, in this case, luminescence brightness is improved.

Preferably, the coelenterazine derivative of the present disclosure is represented by the above general formula (2), where R⁶ is hydrogen. Also, in this case, luminescence brightness is improved.

Particularly preferably, the coelenterazine derivative of the present disclosure is represented by the following structural formula (2-1) or (2-2):

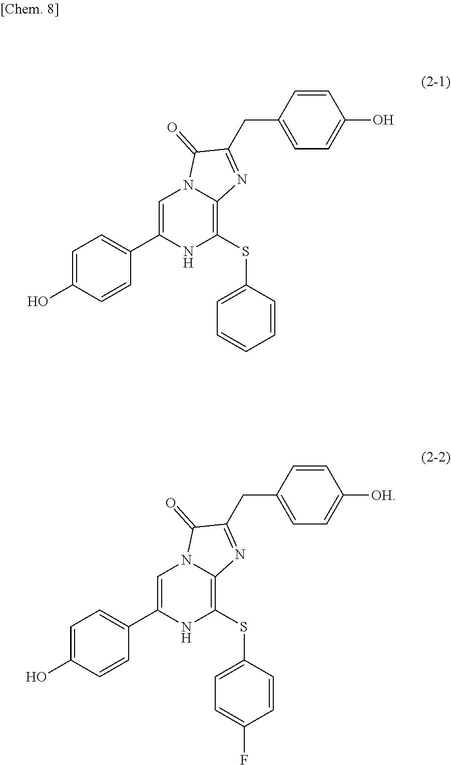 [SMILES: O=c1c(Cc2ccc(O)cc2)nc2c(Sc3ccccc3)[nH]c(-c3ccc(O)cc3)cn1-2 ; O=c1c(Cc2ccc(O)cc2)nc2c(Sc3ccc(F)cc3)[nH]c(-c3ccc(O)cc3)cn1-2]

In this case, the luminescence brightness from the luminescent system using the coelenterazine derivative is particularly high.

### Advantageous Effect

According to the present disclosure, novel coelenterazine derivatives that exhibit high luminescence brightness and have unique enzyme specificity can be provided.

### BRIEF DESCRIPTION OF THE DRAWINGS

In the accompanying drawings:

FIG. 1 is a graph showing the luminescence brightness of each luminescent substrate in live cells using ALuc16 as the luminescent enzyme;

FIG. 2 is a graph showing the luminescence brightness of each luminescent substrate in lysates using ALuc16 as the luminescent enzyme;

FIG. 3 is the luminescence spectra of each luminescent substrate using ALuc16 as the luminescent enzyme;

FIG. 4 is a graph showing the luminescence brightness of each luminescent substrate in live cells using ALuc47 as the luminescent enzyme;

FIG. 5 is a graph showing the luminescence brightness of each luminescent substrate in lysates using ALuc47 as the luminescent enzyme;

FIG. 6 is the luminescence spectra of each luminescent substrate using ALuc47 as the luminescent enzyme;

FIG. 7 is a graph showing the luminescence brightness of each luminescent substrate in live cells using RLuc8.6SG as the luminescent enzyme;

FIG. 8 is a graph showing the luminescence brightness of each luminescent substrate in lysates using RLuc8.6SG as the luminescent enzyme;

FIG. 9 is the luminescence spectra of each luminescent substrate using RLuc8.6SG as the luminescent enzyme;

FIG. 10 is a graph showing the luminescence brightness of each luminescent substrate in live cells using NanoLuc as the luminescent enzyme;

FIG. 11 is a graph showing the luminescence brightness of each luminescent substrate in lysates using NanoLuc as the luminescent enzyme; and

FIG. 12 is the luminescence spectra of each luminescent substrate using NanoLuc as the luminescent enzyme.

### DETAILED DESCRIPTION

Hereinafter, coelenterazine derivatives of the present disclosure are illustrated in detail based on embodiments thereof.

### Coelenterazine Derivative

Coelenterazine derivatives of the present disclosure are represented by the above general formula (1) or (2).

The coelenterazine derivatives of the present disclosure differ from natural coelenterazine (nCTZ) in that the hydroxyphenyl group at position 6 of the imidazopyrazinone skeleton of natural coelenterazine is converted to a bicyclic structure containing oxygen or nitrogen (general formula (1)), or the methylene group in the benzyl group at position 8 is converted to a thioether (general formula (2)). Because of these structural differences, they have unique enzyme specificities different from natural coelenterazine.

Furthermore, the coelenterazine derivatives of the present disclosure maintain high luminescence brightness sufficient for in vivo imaging while having different enzyme specificities from natural coelenterazine and suppressing decay of the luminescence brightness. Thus, they can be used as luminescent substrates for luminescence systems derived from marine organisms.

Without limiting, the luminescence mechanism of the coelenterazine derivatives of the present disclosure when used as luminescent substrates is considered as follows. Similar to natural coelenterazine, the imidazopyrazinone skeleton first becomes an anion state by deprotonation of NH at position 7 by a base, followed by a single electron transfer to triplet oxygen, generating a peroxide anion by radical coupling. This peroxide anion cyclizes to form a dioxetanone intermediate, which decomposes by decarboxylation to generate an excited amidepyrazine in the singlet state, and this amidepyrazine emits light by transitioning to the ground state. Additionally, another pathway is possible depending on the environment of the luminescent enzyme (luciferase). In this pathway, the dioxetanone anion may be protonated, and an excited molecule of neutral amidepyrazine may be generated from neutral dioxetanone, emitting light.

### Coelenterazine Derivative Represented by General Formula (1)

Coelenterazine derivatives of the first embodiment of the present disclosure are represented by the following general formula (1):

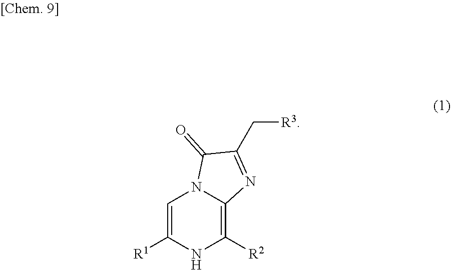

In the above general formula (1), R¹ is represented by the following general formula (1-1-1), (1-1-2), (1-1-3), or (1-1-4):

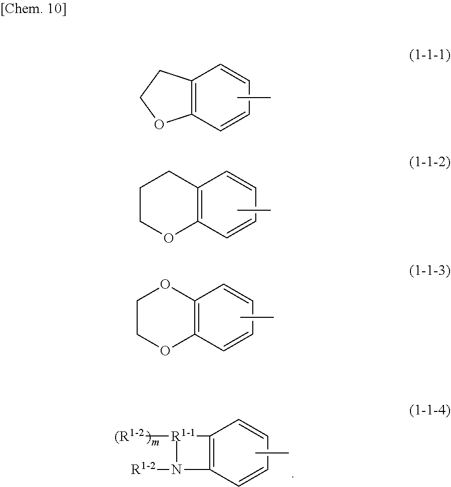

The bonding position of the benzene ring in the general formula (1-1-1), (1-1-2), (1-1-3), or (1-1-4) relative to the position 6 of the imidazopyrazinone skeleton in the general formula (1) is not particularly limited.

In the above general formula (1-1-4), R¹⁻¹ is a hydrocarbon group with 1 to 4 carbon atoms (specifically, a carbon chain), each R¹⁻² is independently hydrogen or a hydrocarbon group with 1 to 3 carbon atoms, and m is an integer from 2 to 8 (specifically, even numbers).

Since R¹⁻¹ in the above general formula (1-1-4) is a hydrocarbon group with 1 to 4 carbon atoms, the group represented by the general formula (1-1-4) has a structure where a 4 to 7-membered ring is fused to the benzene ring. It is preferable that R¹⁻¹ is a linear hydrocarbon group with 1 to 4 carbon atoms.

The hydrocarbon group with 1 to 4 carbon atoms as R¹⁻¹ has m (2 to 8) substituents R¹⁻² bound to it, in other words, R¹⁻¹ is a hydrocarbon group with 1 to 4 carbon atoms and 4 to 10 valences. The substituent R¹⁻² substitutes a hydrogen of the hydrocarbon group. The hydrocarbon group with 1 to 4 carbon atoms as R¹-1 may be either saturated or unsaturated.

When R¹⁻¹ is a hydrocarbon group with 1 carbon atom (when R¹⁻¹ forms a 4-membered ring together with N and the benzene ring), it is preferable that m is 2. As a tetravalent hydrocarbon group with 1 carbon atom, a methane-tetrayl group can be used.

When R¹⁻¹ is a hydrocarbon group with 2 carbon atoms (when R¹⁻¹ forms a 5-membered ring together with N and the benzene ring), it is preferable that m is 2 or 4. In this case, R¹⁻¹ can be either saturated or unsaturated. When R¹⁻¹ is saturated, it is preferable that m is 4, and when R¹⁻¹ is unsaturated, it is preferable that m is 2. As a tetravalent hydrocarbon group (carbon chain) with 2 carbon atoms, an ethene-tetrayl group can be used, and as a hexavalent hydrocarbon group (carbon chain) with 2 carbon atoms, an ethane-hexayl group can be used.

When R¹⁻¹ is a hydrocarbon group with 3 carbon atoms (when R¹⁻¹ forms a 6-membered ring together with N and the benzene ring), it is preferable that m is 2, 4, or 6. In this case, R¹⁻¹ can be either saturated or unsaturated. When R¹⁻¹ is saturated, it is preferable that m is 6, and when R¹⁻¹ is unsaturated, it is preferable that m is 4 or 2. As a tetravalent hydrocarbon group (carbon chain) with 3 carbon atoms, a propyne-tetrayl group can be used. As a hexavalent hydrocarbon group (carbon chain) with 3 carbon atoms, a propene-hexayl group can be used. As an octavalent hydrocarbon group (carbon chain) with 3 carbon atoms, a propane-octayl group can be used.

When R¹⁻¹ is a hydrocarbon group with 4 carbon atoms (when R¹⁻¹ forms a 7-membered ring together with N and the benzene ring), it is preferable that m is 2, 4, 6, or 8. In this case, R¹⁻¹ can be either saturated or unsaturated. When R¹⁻¹ is saturated, it is preferable that m is 8, and when R¹⁻¹ is unsaturated, it is preferable that m is 6, 4, or 2. As a hexavalent hydrocarbon group (carbon chain) with 4 carbon atoms, a butadiene-hexayl group can be used. As an octavalent hydrocarbon group (carbon chain) with 4 carbon atoms, a butene-octayl group can be used. As a decavalent hydrocarbon group with 4 carbon atoms, a butane-decayl group can be used.

Each R¹⁻² in the above general formula (1-1-4) is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R¹ in the above general formula (1) is represented by the above general formula (1-1-3). When R¹ in the general formula (1) is represented by the above general formula (1-1-3), the luminescence brightness is improved.

In the above general formula (1), R² is represented by −R^{2′} or −CH₂—R^{2′}, where R^{2′} is represented by the following general formula (1-2-1), (1-2-2), (1-2-3), (1-2-4), or (1-2-5):

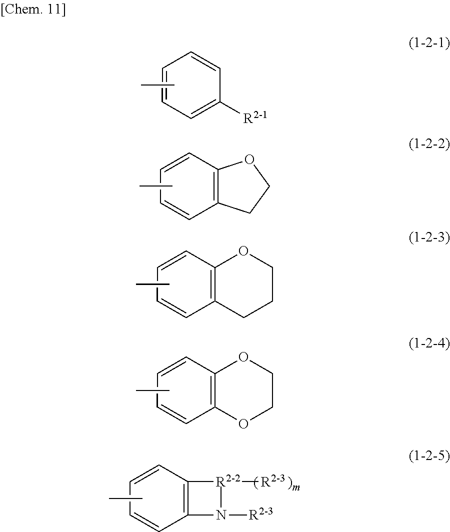

The bonding position of the benzene ring in the general formula (1-2-1), (1-2-2), (1-2-3), (1-2-4), or (1-2-5) relative to the position 8 of the imidazopyrazinone skeleton in the general formula (1) is not particularly limited.

In the above general formula (1-2-1), R²⁻¹ is hydrogen, halogen, —N(R²⁻¹⁻¹)₂, or —OR²⁻¹⁻¹, where each R²⁻¹⁻¹ is independently hydrogen or a hydrocarbon group with 1 to 3 carbon atoms.

In the above general formula (1-2-5), R²⁻² is a hydrocarbon group with 1 to 4 carbon atoms (specifically, a carbon chain), each R²⁻³ is independently hydrogen or a hydrocarbon group with 1 to 3 carbon atoms, and m is an integer from 2 to 8 (specifically, even numbers).

With regard to R²⁻¹ in the above general formula (1-2-1), fluorine, chlorine, bromine, etc., can be used as halogens.

When R²⁻¹ is —N(R²⁻¹⁻¹)₂ or —OR²⁻¹⁻¹, each R²⁻¹⁻¹ is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Since R²⁻² in the above general formula (1-2-5) is a hydrocarbon group with 1 to 4 carbon atoms, the group represented by the general formula (1-2-5) has a structure where a 4 to 7-membered ring is fused to the benzene ring. It is preferable that R²⁻² is a linear hydrocarbon group with 1 to 4 carbon atoms.

The hydrocarbon group with 1 to 4 carbon atoms as R²⁻² has m (2 to 8) substituents R²⁻³ bound to it, in other words, R²⁻² is a hydrocarbon group with 1 to 4 carbon atoms and 4 to 10 valences. The substituent R²⁻³ substitutes a hydrogen of the hydrocarbon group. The hydrocarbon group with 1 to 4 carbon atoms as R²⁻² may be either saturated or unsaturated.

When R²-2 is a hydrocarbon group with 1 carbon atom (when R²⁻² forms a 4-membered ring together with N and the benzene ring), it is preferable that m is 2. As a tetravalent hydrocarbon group with 1 carbon atom, a methane-tetrayl group can be used.

When R²⁻² is a hydrocarbon group with 2 carbon atoms (when R²⁻² forms a 5-membered ring together with N and the benzene ring), it is preferable that m is 2 or 4. In this case, R²⁻² can be either saturated or unsaturated. When R²⁻² is saturated, it is preferable that m is 4, and when R²⁻² is unsaturated, it is preferable that m is 2. As a tetravalent hydrocarbon group (carbon chain) with 2 carbon atoms, an ethene-tetrayl group can be used, and as a hexavalent hydrocarbon group with 2 carbon atoms, an ethane-hexayl group can be used.

When R²⁻² is a hydrocarbon group with 3 carbon atoms (when R²⁻² forms a 6-membered ring together with N and the benzene ring), it is preferable that m is 2, 4, or 6. In this case, R²⁻² can be either saturated or unsaturated. When R²⁻² is saturated, it is preferable that m is 6, and when R²⁻² is unsaturated, it is preferable that m is 4 or 2. As a tetravalent hydrocarbon group (carbon chain) with 3 carbon atoms, a propyne-tetrayl group can be used. As a hexavalent hydrocarbon group (carbon chain) with 3 carbon atoms, a propene-hexayl group can be used. As an octavalent hydrocarbon group (carbon chain) with 3 carbon atoms, a propane-octayl group can be used.

When R²⁻² is a hydrocarbon group with 4 carbon atoms (when R²⁻² forms a 7-membered ring together with N and the benzene ring), it is preferable that m is 2, 4, 6, or 8. In this case, R²⁻² can be either saturated or unsaturated. When R²⁻² is saturated, it is preferable that m is 8, and when R²⁻² is unsaturated, it is preferable that m is 6, 4, or 2. As a hexavalent hydrocarbon group (carbon chain) with 4 carbon atoms, a butadiene-hexayl group can be used. As an octavalent hydrocarbon group (carbon chain) with 4 carbon atoms, a butene-octayl group can be used. As a decavalent hydrocarbon group with 4 carbon atoms, a butane-decayl group can be used.

Each R²⁻³ in the above general formula (1-2-5) is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R² in the above general formula (1) is represented by —CH₂—R^{2′}, and it is preferable that the R^{2′} is represented by the above general formula (1-2-1). When R² in the general formula (1) is represented by—CH₂—R^{2′} and the R^{2′} is represented by the above general formula (1-2-1), the luminescence brightness is improved.

In the above general formula (1), R³ is represented by the following general formula (1-3-1), (1-3-2), or (1-3-3):

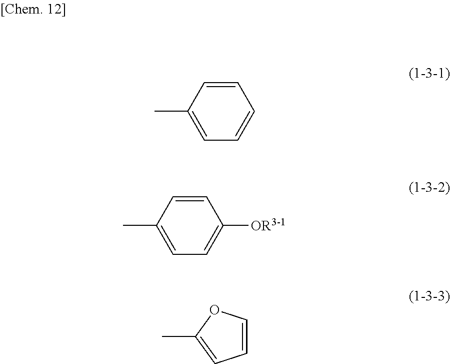

In the above general formula (1-3-2), R³⁻¹ is hydrogen or a hydrocarbon group with 1 to 3 carbon atoms.

With regard to R³⁻¹ in the above general formula (1-3-2), as a

hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R³ in the above general formula (1) is represented by the above general formula (1-3-2). When R³ in the general formula (1) is represented by the above general formula (1-3-2), the luminescence brightness is improved.

In addition, it is preferable that R³⁻¹ in the above general formula (1-3-2) is hydrogen. When R³ in the general formula (1) is represented by the above general formula (1-3-2) and R³⁻¹ is hydrogen, the luminescence brightness is further improved.

It is particularly preferable that the coelenterazine derivative of the present disclosure is represented by the following structural formula (1-1):

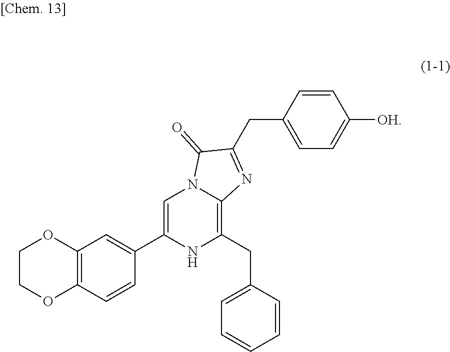 [SMILES: O=c1c(Cc2ccc(O)cc2)nc2c(Cc3ccccc3)[nH]c(-c3ccc4c(c3)OCCO4)cn1-2]

In this case, the luminescence brightness from the luminescent system using the coelenterazine derivative is particularly high.

### Coelenterazine Derivatives Represented by General Formula (2)

Coelenterazine derivatives of the second embodiment of the present disclosure are represented by the following general formula (2):

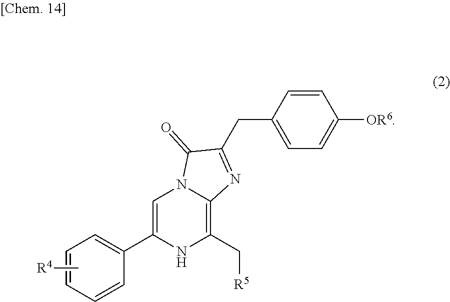

In the general formula (2), R⁴ is hydrogen, —(CH₂)ₙ—OR⁴⁻¹, —N(R⁴⁻¹)₂, or —CF₃, where each R⁴⁻¹ is independently hydrogen or a hydrocarbon group with 1 to 3 carbon atoms, and n is an integer from 0 to 3. In the general formula (2), the bonding position of the R⁴ relative to the benzene ring attached to the position 6 of the imidazopyrazinone skeleton can be o-, m-, or p-.

When R⁴ is —C(CH₂)ₙ—OR⁴⁻¹ or —N(R⁴⁻¹)₂, each R⁴⁻¹ is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R⁴ in the above general formula (2) is represented by —(CH₂)ₙ—OR⁴⁻¹. When R⁴ in the general formula (2) is represented by —(CH₂)ₙ—OR⁴⁻¹, the luminescence brightness is improved.

Furthermore, in —(CH₂)ₙ—OR⁴⁻¹, it is preferable that n is 0, and it is preferable that R⁴-1 is hydrogen. In other words, it is particularly preferable that R⁴ in the above general formula (2) is —OH. When R⁴ in the general formula (2) is —OH, the luminescence brightness is further improved.

In the above general formula (2), R⁵ is represented by the following general formula (2-5-1), (2-5-2), (2-5-3), (2-5-4), or (2-5-5):

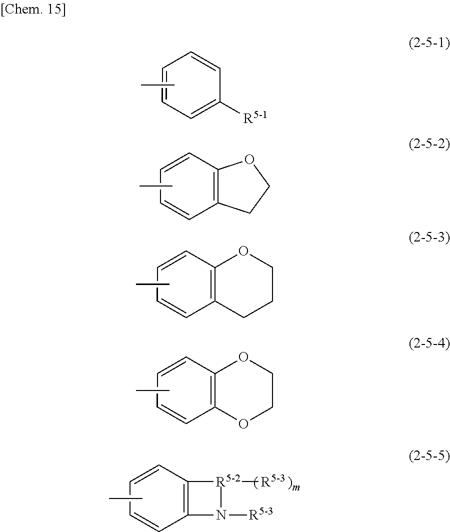

The bonding position of the benzene ring in the general formula (2-5-1), (2-5-2), (2-5-3), (2-5-4), or (2-5-5) relative to S in the general formula (2) is not particularly limited.

In the above general formula (2-5-1), R⁵⁻¹ is hydrogen, halogen, —N(R⁵⁻¹⁻¹)₂, or —OH, where each R⁵⁻¹⁻¹ is independently hydrogen or a hydrocarbon group with 1 to 3 carbon atoms.

In the above general formula (2-5-5), R⁵⁻² is a hydrocarbon group with 1 to 4 carbon atoms (specifically, a carbon chain), each R⁵⁻³ is independently hydrogen or a hydrocarbon group with 1 to 3 carbon atoms, and m is an integer from 2 to 8 (specifically, even numbers).

With regard to R⁵⁻¹ in the above general formula (2-5-1), fluorine, chlorine, bromine, etc., can be used as halogens.

When R⁵⁻¹ is —N(R⁵⁻¹⁻¹)₂, each R⁵⁻¹⁻¹ is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Since R⁵⁻² in the above general formula (2-5-5) is a hydrocarbon group with 1 to 4 carbon atoms, the group represented by the general formula (2-5-5) has a structure where a 4 to 7-membered ring is fused to the benzene ring. It is preferable that R⁵⁻² is a linear hydrocarbon group with 1 to 4 carbon atoms.

The hydrocarbon group with 1 to 4 carbon atoms as R⁵⁻² has m (2 to 8) substituents R⁵⁻³ bound to it, in other words, R⁵⁻² is a hydrocarbon group with 1 to 4 carbon atoms and 4 to 10 valences. The substituent R⁵⁻³ substitutes a hydrogen of the hydrocarbon group. The hydrocarbon group with 1 to 4 carbon atoms as R⁵⁻² may be either saturated or unsaturated.

When R⁵⁻² is a hydrocarbon group with 1 carbon atom (when R⁵⁻² forms a 4-membered ring together with N and the benzene ring), it is preferable that m is 2. As a tetravalent hydrocarbon group with 1 carbon atom, a methane-tetrayl group can be used.

When R⁵⁻² is a hydrocarbon group with 2 carbon atoms (when R⁵⁻² forms a 5-membered ring together with N and the benzene ring), it is preferable that m is 2 or 4. In this case, R⁵⁻² can be either saturated or unsaturated. When R⁵⁻² is saturated, it is preferable that m is 4, and when R²⁻² is unsaturated, it is preferable that m is 2. As a tetravalent hydrocarbon group (carbon chain) with 2 carbon atoms, an ethene-tetrayl group can be used, and as a hexavalent hydrocarbon group with 2 carbon atoms, an ethane-hexayl group can be used.

When R⁵⁻² is a hydrocarbon group with 3 carbon atoms (when R⁵⁻² forms a 6-membered ring together with N and the benzene ring), it is preferable that m is 2, 4, or 6. In this case, R⁵⁻² can be either saturated or unsaturated. When R⁵-2 is saturated, it is preferable that m is 6, and when R⁵⁻² is unsaturated, it is preferable that m is 4 or 2. As a tetravalent hydrocarbon group (carbon chain) with 3 carbon atoms, a propyne-tetrayl group can be used. As a hexavalent hydrocarbon group (carbon chain) with 3 carbon atoms, a propene-hexayl group can be used. As an octavalent hydrocarbon group (carbon chain) with 3 carbon atoms, a propane-octayl group can be used.

When R⁵⁻² is a hydrocarbon group with 4 carbon atoms (when R⁵⁻² forms a 7-membered ring together with N and the benzene ring), it is preferable that m is 2, 4, 6, or 8. In this case, R⁵⁻² can be either saturated or unsaturated. When R⁵⁻² is saturated, it is preferable that m is 8, and when R⁵⁻² is unsaturated, it is preferable that m is 6, 4, or 2. As a hexavalent hydrocarbon group (carbon chain) with 4 carbon atoms, a butadiene-hexayl group can be used. As an octavalent hydrocarbon group (carbon chain) with 4 carbon atoms, a butene-octayl group can be used. As a decavalent hydrocarbon group with 4 carbon atoms, a butane-decayl group can be used.

Each R⁵⁻³ in the above general formula (2-5-5) is independently hydrogen or a hydrocarbon group with 1 to 3 carbon atoms. As a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R⁵ in the above general formula (2) is represented by the above general formula (2-5-1). When R⁵ in the general formula (2) is represented by the above general formula (2-5-1), the luminescence brightness is improved.

Furthermore, R⁵⁻¹ in the above general formula (2-5-1) is preferably hydrogen or halogen, and more preferably hydrogen or fluorine. When R⁵ in the general formula (2) is represented by the general formula (2-5-1) and R⁵⁻¹ is hydrogen or halogen, the luminescence brightness is further enhanced, and when R⁵⁻¹ is hydrogen or fluorine, the luminescence brightness is even further improved.

In the above general formula (2), R⁶ is hydrogen or a hydrocarbon group with 1 to 3 carbon atoms.

With regard to R⁶, as a hydrocarbon group with 1 to 3 carbon atoms, an alkyl group with 1 to 3 carbon atoms, an alkenyl group with 2 to 3 carbon atoms, etc., can be used. As an alkyl group with 1 to 3 carbon atoms, a methyl group, an ethyl group, an n-propyl group, and an isopropyl group can be used. As an alkenyl group with 2 to 3 carbon atoms, a vinyl group, an allyl group, etc., can be used.

Here, it is preferable that R⁶ in the above general formula (2) is hydrogen. When R⁶ in the general formula (2) is hydrogen, the luminescence brightness is improved.

The coelenterazine derivatives of the present disclosure are particularly preferably represented by the following structural formula (2-1) or (2-2):

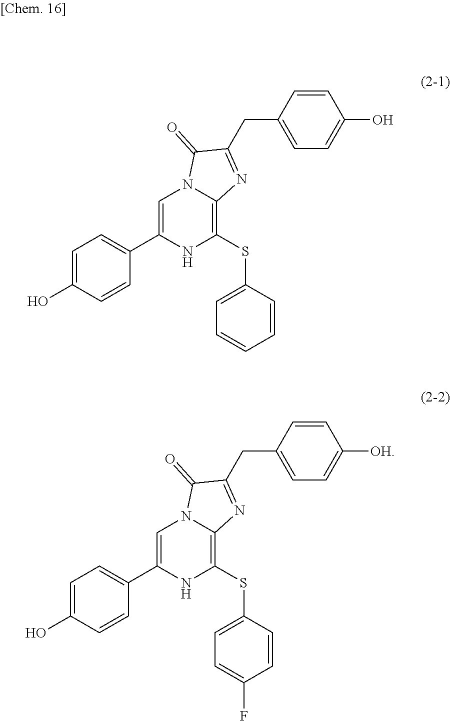 [SMILES: O=c1c(Cc2ccc(O)cc2)nc2c(Sc3ccccc3)[nH]c(-c3ccc(O)cc3)cn1-2 ; O=c1c(Cc2ccc(O)cc2)nc2c(Sc3ccc(F)cc3)[nH]c(-c3ccc(O)cc3)cn1-2]

In this case, the luminescence brightness from the luminescent system using the coelenterazine derivatives is particularly high.

### Synthesis Method of Coelenterazine Derivative Represented by General Formula (1)

Without limitation, the coelenterazine derivatives represented by the above general formula (1) can be synthesized as follows, for example.

(i-1) First, a benzyl compound is synthesized by coupling reaction using 2-amino-3,5-dibromoaminopyrazine, benzylmagnesium chloride, and bis (triphenylphosphine) palladium (II) dichloride.

(i-2) Next, Suzuki-Miyaura coupling is performed using the benzyl compound and boronic acid to synthesize a benzyl compound with the desired ring structure introduced at the position 6.

(ii-1) Alternatively, a phenyl compound is synthesized by Suzuki-Miyaura coupling using 2-amino-3,5-dibromoaminopyrazine and boronic acid.

(ii-2) Next, Suzuki-Miyaura coupling is performed again with the phenyl compound to synthesize a phenyl compound with the desired ring structure introduced at the position 6.

(iii) Finally, the benzyl compound or phenyl compound with the desired ring structure introduced at the position 6 is cyclocondensed with a ketoacetal compound to synthesize the coelenterazine derivative represented by the general formula (1), which is the target substance.

### Synthesis Method of Coelenterazine Derivative Represented by General Formula (2)

Without limitation, the coelenterazine derivative represented by the above general formula (2) can be synthesized as follows, for example.

(i) First, Suzuki-Miyaura coupling is performed using 2-amino-3,5-dibromoaminopyrazine and boronic acid to synthesize an intermediate with the desired ring structure introduced at the position 6.

(ii) Bromination is performed using NBS to synthesize an intermediate brominated at the position 8.

(iii) A substitution reaction is performed using the intermediate brominated at the position 8 and a thiol with sodium hydride to synthesize a thiol compound. Here, optionally, the thiol compound may be reacted with boron tribromide to demethylate it.

(iv) Finally, the thiol compound is cyclocondensed with a ketoacetal compound to synthesize the coelenterazine derivative represented by the general formula (2), which is the target substance.

The coelenterazine derivatives of the present disclosure luminesce through an oxidation reaction catalyzed by marine bioluminescent enzymes.

Therefore, the coelenterazine derivatives of the present disclosure can be used as luminescent labels in biological measurements/detection, for example, to label amino acids, polypeptides, proteins, nucleic acids, etc. The method of coupling the coelenterazine derivatives of the present disclosure to these substances is well-known to those skilled in the art. For example, the coelenterazine derivatives of the present disclosure can be coupled to carboxyl groups or amino groups of the target substances using methods well known to those skilled in the art.

Furthermore, the coelenterazine derivatives of the present disclosure can also be used in measurements/detection utilizing marine bioluminescent enzyme activity generating the luminescence of luminescent substrates. For example, by administering the coelenterazine derivatives of the present disclosure to cells or animals into which marine bioluminescent enzyme genes have been introduced, the expression of target genes or proteins in vivo can be measured/detected.

Among the coelenterazine derivatives of the present disclosure, the coelenterazine derivatives represented by the above general formula (2) can emit light with longer wavelengths than natural coelenterazine, and because light with longer wavelength exerts a higher tissue permeation in living subjects, the coelenterazine derivatives are useful as labeling materials for visualizing deep tissue lesions in vivo.

When the coelenterazine derivatives of the present disclosure are used as luminescent substrates (luciferins), both natural and artificial luminescent enzymes (luciferases) can be utilized.

As natural luminescent enzymes, luciferase derived from sea pansy (Renilla reniformis) (RLuc), luciferase derived from marine copepods (Gaussia princeps) (Gluc), luciferase derived from luminescent shrimp (Oplophorus gracilirostrisk) (Oluc), and luciferase derived from sea cactus can be used.

As artificial luminescent enzymes, “NanoLuc” available from Promega K. K., which is an artificial luminescent enzyme derived from luminescent shrimp (Oplophorus gracilirostris), Renilla luciferase 8.6-535SG (RLuc8.6SG), which is an artificial luminescent enzyme derived from sea pansy (Renilla reniformis), and ALuc, which is a group of artificial luminescent enzymes derived from luminescent plankton developed by Dr. Sung-Bae Kim of the National Institute of Advanced Industrial Science and Technology, can be used. Here, ALuc has various artificial luminescent enzymes, such as ALuc16, ALuc47, and ALuc49, for example.

The coelenterazine derivatives of the present disclosure have luciferase specificity, and the luminescence brightness greatly varies depending on the enzyme. Therefore, the coelenterazine derivatives of the present disclosure enable the simultaneous visualization of multiple in vivo phenomena, such as molecular events occurring in vivo and cancer metastasis, with high sensitivity and speed. For example, they enable to luminesce only a particular luminescent enzyme among many coexisting luminescent enzyme reporters.

These marine bioluminescent enzymes also have the advantage of small molecular size, causing little burden on the organism even when introduced into living subjects, and have high expression efficiency when genes are introduced into living subjects.

The method of introducing genes capable of producing marine

bioluminescent enzymes into living subjects is not particularly limited, and methods using vectors, etc., can be utilized. The production of vectors encoding these marine bioluminescent enzymes is also not particularly limited, and they can be produced by well-known methods. Also, commercially available products can be used as such vectors, for example, “R-luc” available from Promega K. K., “R-luc8” and “R-luc8.6_547” available from Gambhir Lab, Stanford, USA, etc., can be used. Moreover, a plasmid encoding marine bioluminescent enzymes can be transiently expressed in live cells using TransIT-LT1 reagents available from Mirus Bio.

It is preferable that the coelenterazine derivatives of the present disclosure are used as a solution. Here, as solvents used for preparing the solution, alcohols such as methanol and ethanol can be used, besides water. The concentration of the coelenterazine derivatives in the solution can be appropriately selected depending on the purpose, but for example, a range of 1 mM to 5 mM is preferable.

### EXAMPLES

The present disclosure describes in more detail with examples as reference, but the present disclosure is not limited to the following examples in any way.

### Synthesis of Ketoacetal Compound (5)

Commercially available 4-benzyloxybenzyl alcohol (3) (2 g, 9.33 mmol) was dissolved in dry dichloromethane (15 mL), followed by stirring at 0° C. under an argon atmosphere. Thionyl chloride (1.35 mL, 1.64 mmol) was added to the mixture, followed by stirring at room temperature for 2 hours under an argon atmosphere. After adding water to the reaction mixture, extraction was performed with dichloromethane (100 mL×3). The extract was dried over sodium sulfate and concentrated under reduced pressure. The precipitated solid was washed with hexane to obtain compound (4) as a white solid (1.6 g, 6.84 mmol, 74%).

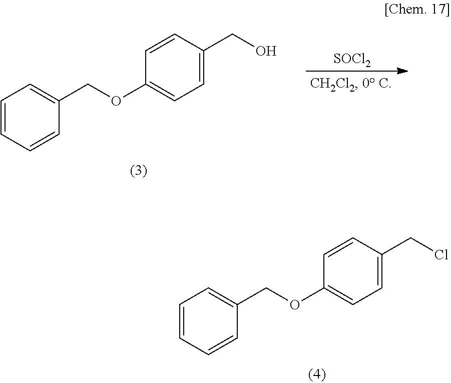 [SMILES: OCc1ccc(OCc2ccccc2)cc1 ; "SOCl2" ; ClCc1ccc(OCc2ccccc2)cc1]

### Identification Result of Compound (4)

¹H-NMR (500 MHZ, CHLOROFORM-D) δ7.43-7.37 (m, 4H), 7.34-7.33 (m, 1H), 7.31 (dd, J=6.6, 2.0 Hz, 2H), 6.95 (dd, J=6.6, 2.0 Hz, 2H), 5.07 (S, 2H), 4.56 (S, 2H)

Magnesium turnings (391 mg, 16.09 mmol) were ground in a mortar and degassed under an argon atmosphere, followed by the addition of dehydrated tetrahydrofuran (10 mL) and 1,2-dibromoethane (0.2 mL), and stirring for 1 hour. The reaction mixture was cooled and then heated under reflux for 2 hours after adding compound (4) (1.5 g, 6.45 mmol) dissolved in dehydrated tetrahydrofuran (10 mL). The reaction mixture (Grignard reagent) was then cooled on ice. After dissolving diethoxyethyl acetate (1.72 mL, 9.67 mmol) in dehydrated tetrahydrofuran (10 mL) and cooling the solution to −80° C., the entire amount of the Grignard reagent was added dropwise over 20 minutes, followed by stirring for 3 hours at −80° C. The mixture was quenched with water while keeping it cool, returned to room temperature, and extraction was performed with ethyl acetate (200 mL×3).

The extract was dried over sodium sulfate and concentrated under reduced pressure. The residue was dissolved in ethanol (30 mL), and palladium on carbon (160 mg) was added, followed by stirring under a hydrogen atmosphere at room temperature for 12 hours. The reaction mixture was filtered through Celite, and concentrated under reduced pressure. The residue was purified by a silica gel chromatography (w=65 g, ϕ=4.0 cm, hexane: ethyl acetate=4:1→3:1) to obtain ketoacetal compound (5) as a light yellow oil (669 mg, 2.81 mmol, 43%).

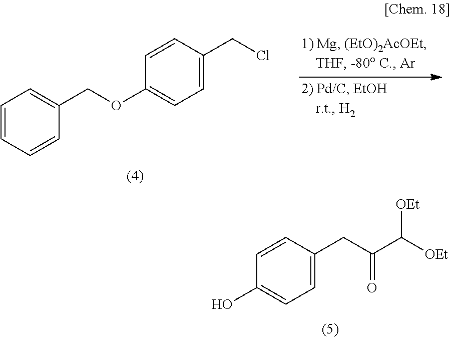 [SMILES: ClCc1ccc(OCc2ccccc2)cc1 ; CCOC(OCC)C(=O)Cc1ccc(O)cc1]

### Identification of Ketoacetal Compound (5)

¹H-NMR (500 MHZ, CHLOROFORM-D) δ7.05 (dd, J=11.5, 2.9 Hz, 2H), 6.75 (dd, J=11.5, 3.4 Hz, 2H), 5.58 (S, 1H), 4.65 (S, 1H), 3.82 (S, 2H), 3.73-3.67 (m, 2H), 3.58-3.52 (m, 2H), 1.24 (t, J=7.2 Hz, 6H)

HR-ESI-MS: m/z: [M+H]⁺ calculated for C₁₃H₁₈Na₁O₄: 261.11141; found: 261.11028

### Synthesis of the Compound Represented by Structural Formula (1-1)

Zinc chloride (2.7 g, 19.77 mmol) and 1 M benzylmagnesium chloride solution in tetrahydrofuran (22 mL, 19.77 mmol) were stirred for 1 hour under an argon atmosphere. Bis(triphenylphosphine)palladium (II) dichloride (351 mg, 0.49 mmol) dissolved in dehydrated tetrahydrofuran (22 mL) and 2-amino-3,5-dibromopyrazine (6) (2.5 g, 9.89 mmol) were added to this mixture, followed by stirring at room temperature for 4 days. After adding water to the reaction mixture, extraction was performed with ethyl acetate (200 mL×3). The extract was dried over sodium sulfate and concentrated under reduced pressure. The residue was purified by a silica gel chromatography (w=170 g, ϕ=4.0 cm, hexane: ethyl acetate=4:1→3:1) to obtain compound (7) as a yellow oil (2.1 g, 7.98 mmol, 81%).

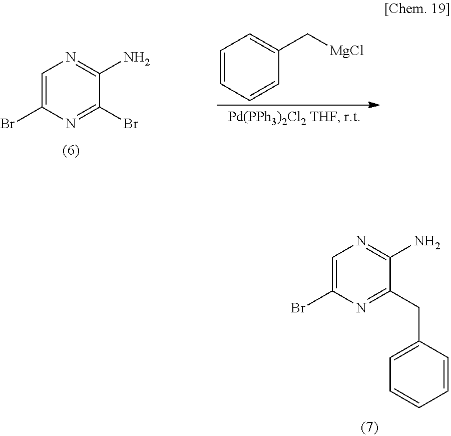

### Identification Results of Compound (7)

¹H-NMR (500 MHZ, CHLOROFORM-D) δ8.03 (s, 1H), 7.34-7.21 (m, 5H), 4.41 (s, 2H), 4.08 (s, 2H)

HR-ESI-MS: m/z: [M+H]⁺ calculated for C₁₁H₁₁⁷⁹Br₁N₃: 264.01363; found: 264.01253

HR-ESI-MS: m/z: [M+H]⁺ calculated for C₁₁H₁₁⁸¹Br₁N₃: 266.01103; found: 266.01159

Compound (7) (150 mg, 0.567 mmol) and commercially available 1,4-benzodioxane-6-boronic acid (133 mg, 0.741 mmol) were dissolved in 1,4-dioxane (5 mL), degassed in a simplified manner, and then placed under an argon atmosphere. Tetrakis(triphenylphosphine)palladium(0) (32 mg, 0.028 mmol) and 2M sodium carbonate aqueous solution (5 mL) were added to this mixture, followed by stirring at 110° C. for 1.5 hours. After returning the reaction mixture to room temperature, extraction was performed with ethyl acetate (60 mL×2). The extract was dried over sodium sulfate and concentrated under reduced pressure. The residue was purified by a silica gel chromatography (w=60 g, ϕ=3.0 cm, hexane: ethyl acetate=1:1→ethyl acetate) to obtain compound (8) as a pale yellow solid (142 mg, 0.444 mmol, 78%).

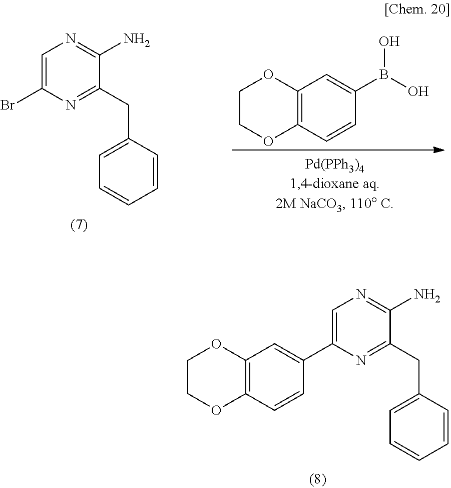 [SMILES: Nc1ncc(Br)nc1Cc1ccccc1 ; OB(O)c1ccc2c(c1)OCCO2 ; Nc1ncc(-c2ccc3c(c2)OCCO3)nc1Cc1ccccc1]

### Identification Results of Compound (8)

¹H-NMR (500 MHz, ACETONE-D6) δ8.35 (s, 1H), 7.48 (d, J=2.3 Hz, 1H), 7.45 (dd, J=8.3, 2.0 Hz, 1H), 7.37 (d, J=8.0 Hz, 2H), 7.30 (t, J=7.7 Hz, 2H), 7.21 (t, J=7.4 Hz, 1H), 6.87 (d, J=8.6 Hz, 1H), 5.54 (s, 1H), 4.29 (s, 4H), 4.16 (s, 2H)

HR-ESI-MS: m/z: [M+H]⁺ calculated for C₁₉H₁₈N₃O₂: 320.13956; found: 320.13990

Substrate (8) (30 mg, 0.094 mmol) and ketoacetal compound (5) (33 mg, 0.14 mmol) were dissolved in ethanol (2 mL), and 12 M hydrochloric acid (100 μL) was added, followed by stirring at 60° C. for 12 hours. The reaction mixture was concentrated under reduced pressure and the resultant was purified by an automated preparative medium-pressure chromatography (chloroform: methanol=99:1→85:15) to obtain the compound represented by structural formula (1-1) as a yellow solid (26 mg, 0.056 mmol, 60%).

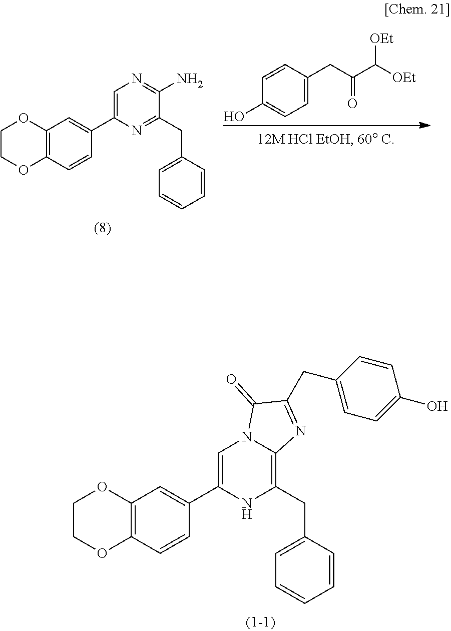 [SMILES: Nc1ncc(-c2ccc3c(c2)OCCO3)nc1Cc1ccccc1 ; CCOC(OCC)C(=O)Cc1ccc(O)cc1 ; O=c1c(Cc2ccc(O)cc2)nc2c(Cc3ccccc3)[nH]c(-c3ccc4c(c3)OCCO4)cn1-2]

### Identification Results of Compound Represented by Structural Formula (1-1)

¹H-NMR (500 MHZ, METHANOL-D4, 0.5% TFA-D) δ8.39 (s, 1H), 7.44 (d, J=1.7 Hz, 1H), 7.41-7.38 (m, 3H), 7.32-7.29 (m, 2H), 7.26-7.23 (m, 1H), 7.10 (dd, J=11.5, 2.9 Hz, 2H), 6.95 (d, J=8.6 Hz, 1H), 6.73 (dd, J=11.5, 2.9 Hz, 2H), 4.52 (s, 2H), 4.29 (m, 4H), 4.17 (s, 2H)

HR-ESI-MS: m/z: [M+H]⁺ calculated for C₂₈H₂₄N₃O₄: 466.17655; found: 466.17668

### Synthesis of the Compound Represented by Structural Formula (2-1)

Commercially available 2-amino-5-dibromopyrazine (9) (2.0 g, 11.49 mmol) and commercially available 4-methoxyphenylboronic acid (2.6 g, 17.24 mmol) were dissolved in 1,4-dioxane (30 mL), degassed in a simplified manner, and then placed under an argon atmosphere. Tetrakis(triphenylphosphine)palladium(0) (654 mg, 0.86 mmol) and 2 M sodium carbonate aqueous solution (30 mL) were added to this mixture, followed by stirring at 110° C. for 1.5 hours. After returning the reaction mixture to room temperature, water was added and extraction was performed with ethyl acetate (200 mL+2). The extract was dried over sodium sulfate and concentrated under reduced pressure. The residue was purified by a silica gel chromatography (w=100 g, ϕ=4.0 cm, hexane: ethyl acetate=2:1→1:1) to obtain compound (10) as a light yellow solid (2.62 g, 13.03 mmol, 113%).

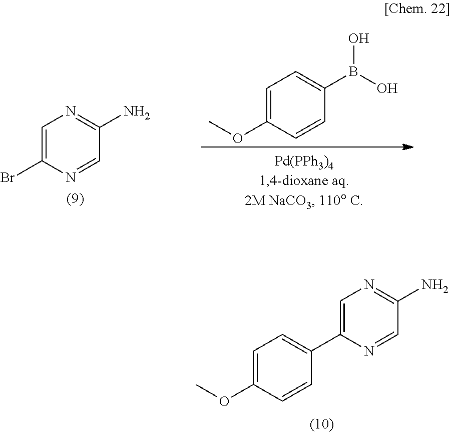

### Identification Results of Compound (10)

¹H-NMR (500 MHZ, CHLOROFORM-D) δ8.40 (d, J=1.7 Hz, 1H), 8.04 (d, J=1.7 Hz, 1H), 7.81 (td, J=6.0, 3.4 Hz, 2H), 6.98 (td, J=6.0, 3.4 Hz, 2H), 4.54 (s, 2H), 3.86 (s, 3H)

HR-ESI-MS: m/z: [M+H]⁺ calculated for C₁₆H₁₄N₃O₁: 202.09755; found: 202.09804

Compound (10) (2.62 g, 13.02 mmol) was dissolved in chloroform (70 mL), and N-bromosuccinimide (3.01 g, 16.93 mmol) was then added, followed by stirring at room temperature for 1 hour. The reaction mixture was cooled in an ice bath, quenched with water, and extraction was performed with chloroform (100 mL×2). The extract was dried over sodium sulfate and concentrated under reduced pressure. The residue was purified by a silica gel chromatography (w=50 g, ϕ=4.0 cm, hexane: ethyl acetate=3:1→2:1) to obtain compound (11) as a reddish-brown solid (2.04 g, 7.31 mmol, 56%).

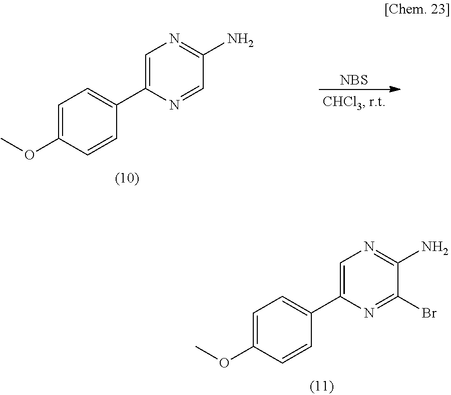 [SMILES: COc1ccc(-c2cnc(N)cn2)cc1 ; "NBS" ; COc1ccc(-c2cnc(N)c(Br)n2)cc1]

### Identification Results of Compound (11)

¹H-NMR (500 MHZ, CHLOROFORM-D) δ8.34 (s, 1H), 7.81 (td, J=6.0, 3.4 Hz, 2H), 6.97 (td, J=6.0, 3.4 Hz, 2H), 4.99 (s, 2H), 3.85 (s, 3H)

HR-ESI-MS: m/z: [M+H]⁺ calculated for C₁₁H₁₁⁷⁹Br₁N₃O₁: 280.00907; found: 280.00855

HR-ESI-MS: m/z: [M+H]⁺ calculated for C₁₁H₁₁⁸¹Br₁N₃: 282.00530; found: 251.00650

Benzene thiol (273 μL, 2.68 mmol) was dissolved in dry N,N-dimethylformamide (20 mL), which was cooled in an ice bath, and sodium hydride (178 mg, 4.46 mmol) was then added, followed by stirring at 0° C. under an argon atmosphere for 1 hour. Compound (11) (500 mg, 1.78 mmol) was added to this mixture, followed by stirring at 100°° C. for 2 hours. After returning the reaction mixture to room temperature, water was added and extraction was performed with ethyl acetate (200 mL×3). The extract was dried over sodium sulfate, toluene was added, and the resultant was concentrated under reduced pressure. The residue was purified by a silica gel chromatography (w=90 g, ϕ=3.0 cm, hexane: ethyl acetate=2:1→1:1) to obtain compound (12) as a brown solid (441 mg, 1.43 mmol, 80%).

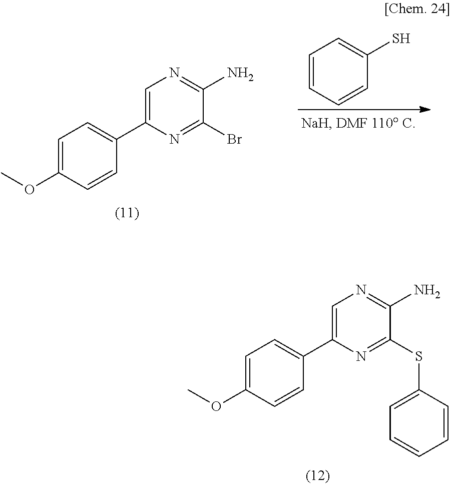 [SMILES: COc1ccc(-c2cnc(N)c(Br)n2)cc1 ; Sc1ccccc1 ; COc1ccc(-c2cnc(N)c(Sc3ccccc3)n2)cc1]

### Identification Results of Compound (12)

¹H-NMR (500 MHZ, CHLOROFORM-D) δ8.30 (s, 1H), 7.71 (dd, J=6.6, 2.0 Hz, 2H), 7.49 (dd, J=8.3, 1.4 Hz, 2H), 7.40-7.35 (m, 3H), 6.91 (dd, J=6.9, 2.3 Hz, 2H), 4.84 (s, 2H), 3.83 (s, 3H)

HR-ESI-MS: m/z: [M+H]⁺ calculated for C₁₇H₁₆N₃O₁S₁: 310.10158; found: 310.10141

Compound (12) (150 mg, 0.48 mmol) was placed under an argon atmosphere, dehydrated dichloromethane (8 mL) was added, and the resulting mixture was cooled to −80° C. Boron tribromide (4.8 mL, 4.85 mmol) was added to the reaction mixture, and the reaction mixture was returned to room temperature and stirred for 12 hours. The reaction mixture was cooled in an ice bath, quenched with saturated sodium bicarbonate aqueous solution, and extraction was performed with chloroform (50 mL×3). The extract was dried over sodium sulfate and concentrated under reduced pressure. The residue was purified by a silica gel chromatography (w=50 g, ϕ=2.0 cm, hexane: ethyl acetate 32 3:1→1:1) to obtain compound (13) as a reddish-brown solid (115 mg, 0.39 mmol, 81%).

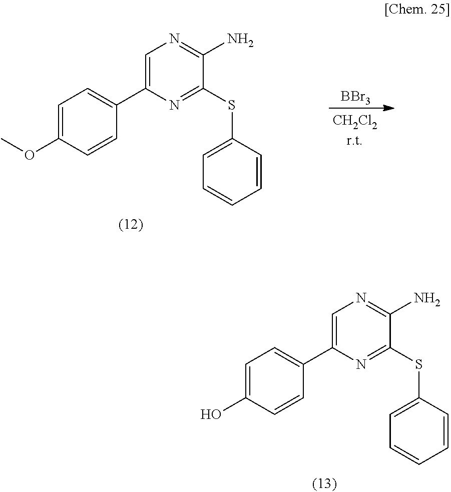 [SMILES: COc1ccc(-c2cnc(N)c(Sc3ccccc3)n2)cc1 ; "BBr3" ; Nc1ncc(-c2ccc(O)cc2)nc1Sc1ccccc1]

### Identification Results of Compound (13)

¹H-NMR (500 MHZ, ACETONE-D6) δ8.31 (s, 1H), 7.64 (dd, J=6.9, 2.3 Hz, 2H), 7.51 (dt, J=8.2, 1.9 Hz, 2H), 7.42-7.35 (m, 3H), 6.80 (dd, J=6.6, 2.0 Hz, 2H), 5.75 (s, 2H)

HR-ESI-MS: m/z: [M+H]⁺ calculated for C₁₆H₁₄N₃O₁S₁: 296.08613; found: 296.08576

Compound (13) (30 mg, 0.101 mmol) and ketoacetal compound (5) (36 mg, 0.153 mmol) were dissolved in ethanol (2 mL), and 12 M hydrochloric acid (100 μL) was then added, followed by stirring at 60° C. for 12 hours. The reaction mixture was concentrated under reduced pressure and the resultant was purified by an automated medium-pressure column chromatography (chloroform: methanol=99:1→85:15) to obtain the compound represented by structural formula (2-1) as a reddish-brown solid (23 mg, 0.052 mmol, 51%).

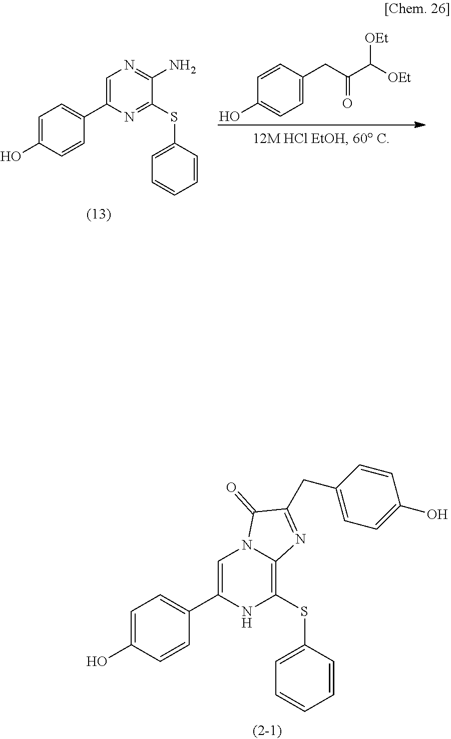 [SMILES: Nc1ncc(-c2ccc(O)cc2)nc1Sc1ccccc1 ; CCOC(OCC)C(=O)Cc1ccc(O)cc1 ; O=c1c(Cc2ccc(O)cc2)nc2c(Sc3ccccc3)[nH]c(-c3ccc(O)cc3)cn1-2]

### Identification Results of Compound Represented by Structural Formula (2-1)

¹H-NMR (500 MHZ, METHANOL-D4) δ8.16 (s, 1H), 7.66 (dd, J=7.2, 2.0 Hz, 2H), 7.53-7.49 (m, 5H), 7.11 (d, J=8.6 Hz, 2H), 6.72 (d, J=4.6 Hz, 2H), 6.70 (d, J=4.0 Hz, 2H), 4.03 (s, 2H)

HR-ESI-MS: m/z: [M+H]⁺ calculated for C₂₅H₂₀N₃O₃S₁: 442.12153; found: 442.12254

Synthesis of the compound Represented by Structural Formula (2-2)

Compound (11) was synthesized in the same manner as in the section of the synthesis of the compound represented by structural formula (2-1).

4-Fluorobenzene thiol (285 μL, 2.14 mmol) was dissolved in dry N,N-dimethylformamide (20 mL), which was cooled in an ice bath, and sodium hydride (178 mg, 4.46 mmol) was then added, followed by stirring at 0° C. under an argon atmosphere for 1 hour. Compound (11) (500 mg, 1.78 mmol) was added to this mixture, followed by stirring at 100° C. for 2.5 hours. After returning the reaction mixture to room temperature, water was added and extraction was performed with ethyl acetate (200 mL×3). The extract was dried over sodium sulfate, toluene was added, and the resultant was concentrated under reduced pressure. The residue was purified by a silica gel chromatography (w=95 g, ϕ=3.0 cm, chloroform: ethyl acetate=2:1→1:1) to obtain compound (14) as a pale orange solid (396 mg, 1.21 mmol, 68%).

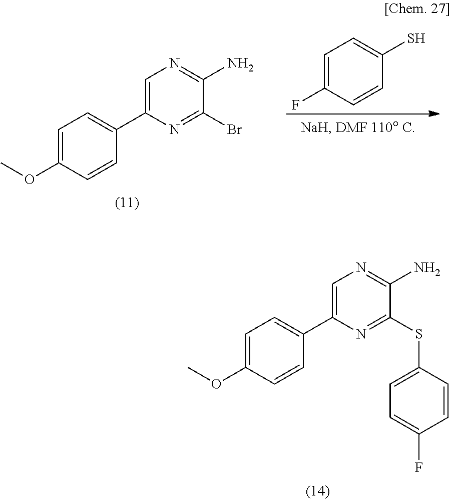 [SMILES: COc1ccc(-c2cnc(N)c(Br)n2)cc1 ; Fc1ccc(S)cc1 ; COc1ccc(-c2cnc(N)c(Sc3ccc(F)cc3)n2)cc1]

### Identification Results of Compound (14)

¹H-NMR (500 MHZ, CHLOROFORM-D) δ8.26 (s, 1H), 7.65 (dd, J=6.9, 2.3 Hz, 2H), 7.54-7.51 (m, 2H), 7.12-7.08 (m, 2H), 6.89 (dd, J=6.9, 1.7 Hz, 2H), 4.89 (s, 2H), 3.82 (s, 3H)

HR-ESI-MS: m/z: [M+H]⁺ calculated for C₁₇H₁₅F₁N₃O₁S₁: 328.09432; found: 328.09199

Compound (14) (50 mg, 0.15 mmol) was placed under an argon atmosphere, dehydrated dichloromethane (5 mL) was added, and the resulting mixture was cooled to −80° C. Boron tribromide (0.9 mL, 1.21 mmol) was added to the reaction mixture, and the solution mixture was returned to room temperature and stirred for 12 hours. The reaction mixture was cooled in an ice bath, quenched with saturated sodium bicarbonate aqueous solution, and extraction was performed with chloroform (15 mL×3). The extract was dried over sodium sulfate and concentrated under reduced pressure. The residue was purified by a silica gel chromatography (w=30 g, ϕ=1.5 cm, hexane: ethyl acetate=3:1→1:1) to obtain compound (15) as a yellow solid (38 mg, 0.12 mmol, 76%).

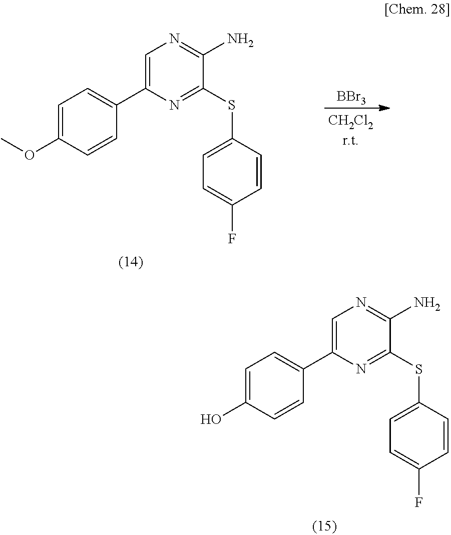 [SMILES: COc1ccc(-c2cnc(N)c(Sc3ccc(F)cc3)n2)cc1 ; "BBr3" ; Nc1ncc(-c2ccc(O)cc2)nc1Sc1ccc(F)cc1]

### Identification Results of Compound (15)

¹H-NMR (500 MHZ, METHANOL-D4) δ8.15 (s, 1H), 7.57 (td, J=5.9, 2.5 Hz, 2H), 7.50 (dd, J=6.6, 2.0 Hz, 2H), 7.20-7.16 (m, 2H), 6.73 (dd, J=6.6, 2.0 Hz, 2H)

HR-ESI-MS: m/z: [M+H]⁺ calculated for C₁₆H₁₃F₁N₃O₁S₁: 314.07754; found: 314.07634

Compound (15) (30 mg, 0.096 mmol) and ketoacetal compound (5) (34 mg, 0.14 mmol) were dissolved in ethanol (2 mL), and 12 M hydrochloric acid (100 μL) was then added, followed by stirring at 60° C. for 12 hours. The reaction mixture was concentrated under reduced pressure, and the resultant was purified by automatic medium-pressure column chromatography (chloroform: methanol=99:1→85:15) to obtain the compound represented by structural formula (2-2) as a brown solid (22 mg, 0.049 mmol, 51%).

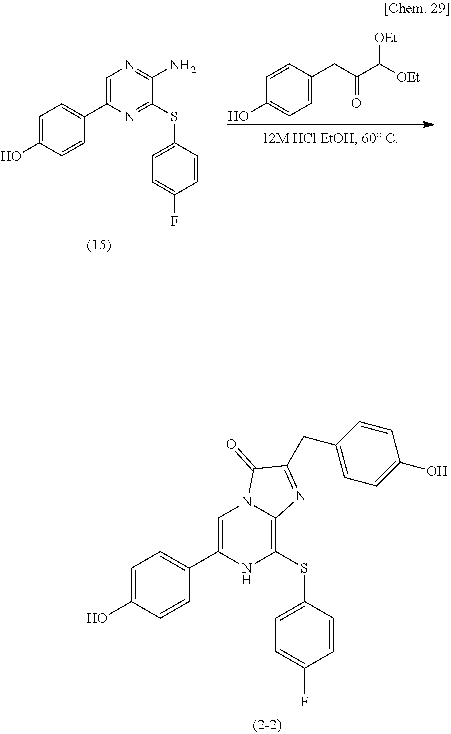 [SMILES: Nc1ncc(-c2ccc(O)cc2)nc1Sc1ccc(F)cc1 ; CCOC(OCC)C(=O)Cc1ccc(O)cc1 ; O=c1c(Cc2ccc(O)cc2)nc2c(Sc3ccc(F)cc3)[nH]c(-c3ccc(O)cc3)cn1-2]

### Identification Results of Compound Represented by Structural Formula (2-2)

¹H-NMR (500 MHz, METHANOL-D4) δ8.10 (s, 1H), 7.65 (qd, J=5.7, 3.0 Hz, 2H), 7.46 (dd, J=6.9, 2.3 Hz, 2H), 7.24-7.21 (m, 2H), 7.10 (d, J=8.0 Hz, 2H), 6.71 (d, J=8.6 Hz, 2H), 6.69 (dd, J=6.6, 2.0 Hz, 2H), 4.02 (s, 2H)

HR-ESI-MS: m/z: [M+H]⁺ calculated for C₂₅H₁₉F₁N₃O₃S₁: 460.11304; found: 460.11311

### Synthesis of Comparative Compound (17)

Compound (7) was synthesized in the same manner as in the section of the synthesis of the compound represented by structural formula (1-1).

Comparative compound (17) was synthesized in the same manner as the synthesis of the compound represented by structural formula (1-1) by using 3-pyridylboronic acid instead of 1,4-benzodioxane-6-boronic acid.

The reaction scheme is shown below.

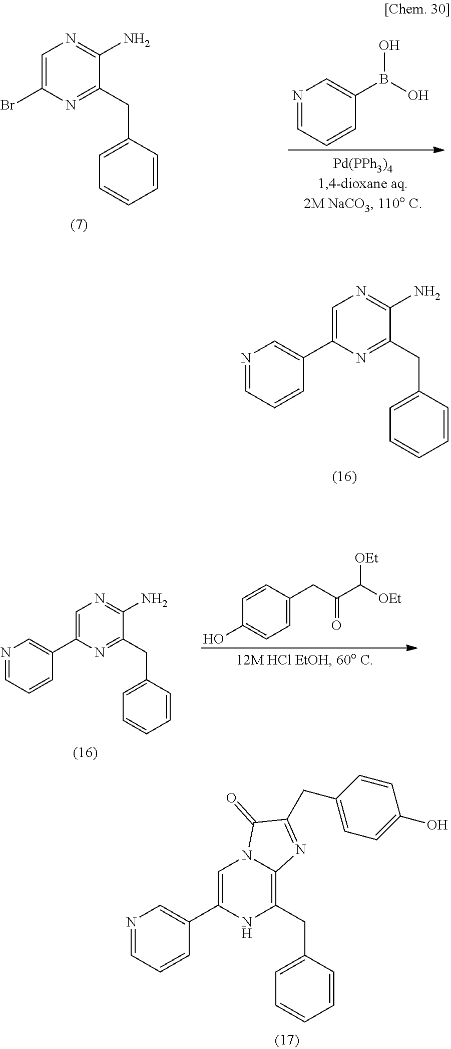 [SMILES: Nc1ncc(Br)nc1Cc1ccccc1 ; OB(O)c1cccnc1 ; Nc1ncc(-c2cccnc2)nc1Cc1ccccc1 ; Nc1ncc(-c2cccnc2)nc1Cc1ccccc1 ; CCOC(OCC)C(=O)Cc1ccc(O)cc1 ; O=c1c(Cc2ccc(O)cc2)nc2c(Cc3ccccc3)[nH]c(-c3cccnc3)cn1-2]

### Identification Results of Comparative Compound (17)

¹H-NMR (500 MHZ, METHANOL-D4) δ8.96 (s, 1H), 8.57 (dd, J=4.9, 1.4 Hz, 1H), 8.21 (d, J=8.0 Hz, 1H), 8.06 (s, 1H), 7.52 (dd, J=8.0, 5.2 Hz, 1H), 7.39 (d, J=7.4 Hz, 2H), 7.28 (t, J=7.4 Hz, 2H), 7.22-7.19 (m, 1H), 7.13 (d, J=8.6 Hz, 2H), 6.69 (d, J=8.6 Hz, 2H), 4.41 (s, 2H), 4.07 (s, 2H)

HR-ESI-MS: m/z: [M+H]⁺ calculated for C₂₅H₂₁N₄O₂: 409.16635; found: 409.16645

### Synthesis of Comparative Compound (19)

Compound (7) was synthesized in the same manner as in the section of the synthesis of the compound represented by structural formula (1-1).

Comparative compound (19) was synthesized in the same manner as the synthesis of the compound represented by structural formula (1-1) by using 4-pyridylboronic acid instead of 1,4-benzodioxane-6-boronic acid.

The reaction scheme is shown below.

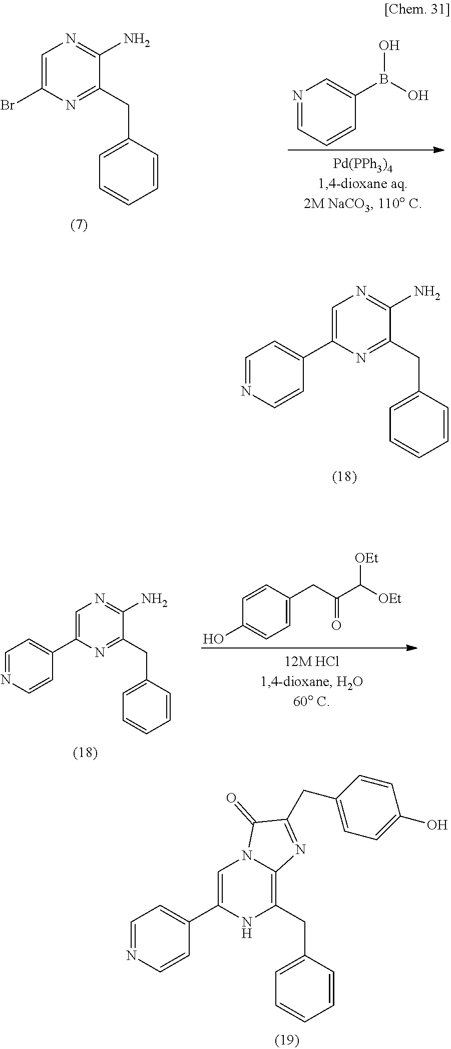 [SMILES: Nc1ncc(Br)nc1Cc1ccccc1 ; OB(O)c1cccnc1 ; Nc1ncc(-c2ccncc2)nc1Cc1ccccc1 ; Nc1ncc(-c2ccncc2)nc1Cc1ccccc1 ; CCOC(OCC)C(=O)Cc1ccc(O)cc1 ; O=c1c(Cc2ccc(O)cc2)nc2c(Cc3ccccc3)[nH]c(-c3ccncc3)cn1-2]

Identification results of Comparative Compound (19)

¹H-NMR (500 MHZ, METHANOL-D4) δ9.17 (s, 1H), 8.90 (d, J=6.9 Hz, 2H), 8.72 (d, J=6.9 Hz, 2H), 7.42 (d, J=6.9 Hz, 2H), 7.31-7.28 (m, 2H), 7.24-7.21 (m, 1H), 7.11 (dd, J=11.5, 2.9 Hz, 2H), 6.71 (dd, J=6.6, 2.0 Hz, 2H), 4.55 (s, 2H), 4.17 (s, 2H)

HR-ESI-MS: m/z: [M+H]⁺ calculated for C₂₅H₂₁N₄O₂: 409.16614; found: 409.16645

### Synthesis of Comparative Compound (22)

Comparative compound (22) was synthesized in the same manner as the synthesis of the compound represented by structural formula (1-1) by using phenylboronic acid instead of benzylmagnesium chloride and 4-hydroxyboronic acid instead of 1,4-benzodioxane-6-boronic acid.

The reaction scheme is shown below.

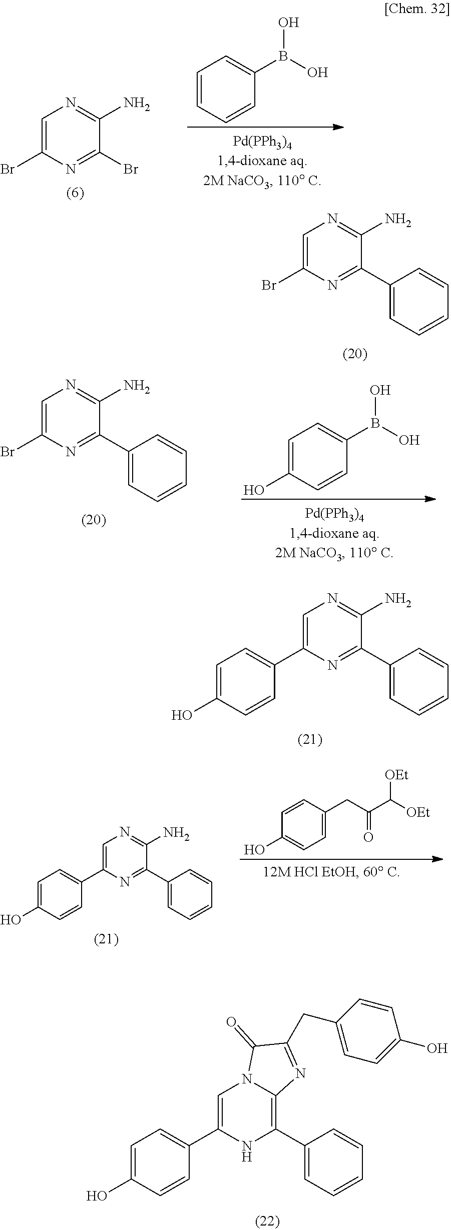

Identification results of Comparative Compound (22)

¹H-NMR (500 MHZ, METHANOL-D4) δ8.01 (d, J=5.2 Hz, 2H), 7.65 (s, 1H), 7.60-7.55 (m, 3H), 7.13 (d, J=8.6 Hz, 2H), 6.90 (dd, J=6.6, 2.0 Hz, 2H), 6.66 (dd, J=6.6, 2.0 Hz, 2H), 4.03 (s, 2H)

HR-ESI-MS: m/z: [M+H]⁺ calculated for C₂₅H₂₀N₃O₃: 410.14906; found: 410.15047

Additionally, the following natural substrates (nCTZ), the known natural analog substrate 1 (CTZh, Coelenterazine h), and the natural analog 10 substrate 2 (DBC, DeepBlueC) were used as other comparative substrates.

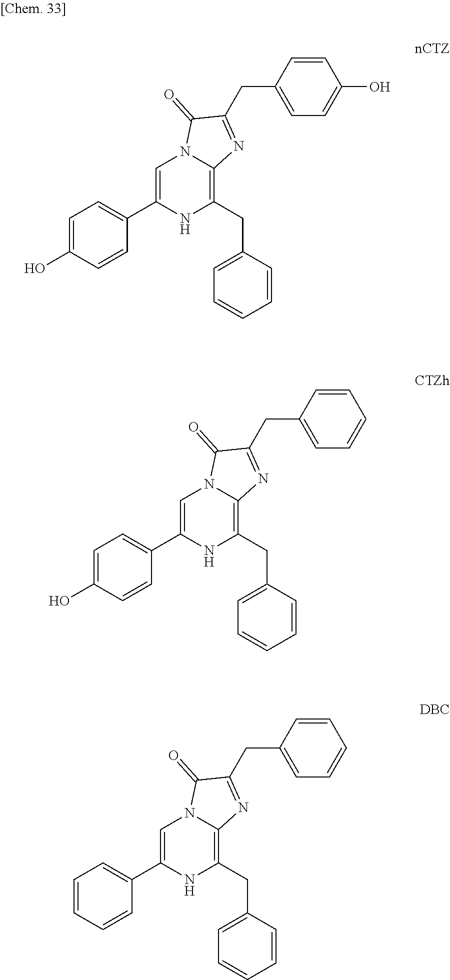 [SMILES: O=c1c(Cc2ccc(O)cc2)nc2c(Cc3ccccc3)[nH]c(-c3ccc(O)cc3)cn1-2 ; O=c1c(Cc2ccccc2)nc2c(Cc3ccccc3)[nH]c(-c3ccc(O)cc3)cn1-2 ; O=c1c(Cc2ccccc2)nc2c(Cc3ccccc3)[nH]c(-c3ccccc3)cn1-2]

### Luminescence Spectrum Measurement

Measurements were conducted using the luminescence spectrum device AB-1850 available from ATTO Corporation.

The measured spectra were obtained with a measurement parameter-tuned detector.

### Luminescence Measurement in Live Cells and Lysate Cells

For luminescence measurements, the IVIS Imaging System purchased from PerkinElmer (old name: Caliper Life Sciences) was used.

As live cells expressing luminescent enzymes, COS-7 cells derived from the kidney of an African green monkey were used, and the evaluations were also conducted with the lysates produced by the lysis of these live cells.

### Evaluation of Luminescence Brightness

The COS-7 cells derived from the kidney of the African green monkey were seeded in a 6-well microplate and were cultured in a CO₂ incubator until 70% of the bottom area was covered by the cells. Using TransIT-LT1 reagent (Mirus), the plasmid encoding each of the following marine bioluminescent enzymes was transiently expressed in cells in each well.

(i) ALuc16

(ii) ALuc47

(iii) Renilla luciferase 8.6-535SG (RLuc8.6SG)

(iv) NanoLuc

After this lipofection, the cells were cultured in a CO₂ incubator for one day. The cells were then subcultured into a 96-well microplate and further incubated for one day.

The wells with cells transfected with the same plasmid were conceptually divided into two, where one was used for lysate experiments and the other for live cell experiments. For the lysate, first, the cell medium was removed from the 96-well microplate, and then 40 μL of cell lysis reagent (lysate) available from Promega K. K. was injected into each well and the plate was incubated for 15 minutes. For the live cell experiments, after the cell medium was completely removed, the microplate was immediately sealed to prevent the sample evaporation until the luminescence measurements.

Each luminescent substrate was first dissolved in methanol (PEG400, 25%) to 5 mM (stock solution), which was further diluted with phosphate-buffered saline (PBS) to 100 μM (this solution is hereinafter referred to as diluted luminescent substrate solution). This “diluted luminescent substrate solution” was dispensed in advance into each empty well of a 96-well microplate. To the 96-well black frame microplate containing the aforementioned cell lysis solution (lysate), 40 μL of the “diluted luminescent substrate solution” was simultaneously injected using a 12-channel micropipette. Immediately after the diluted luminescent substrate solution was injected, the microplate was transferred to the IVIS Imaging System (Xenogen, USA). The bioluminescence brightness from the 96-well black frame microplate was measured, and analyzed using the dedicated software Living Image ver. 4.7.

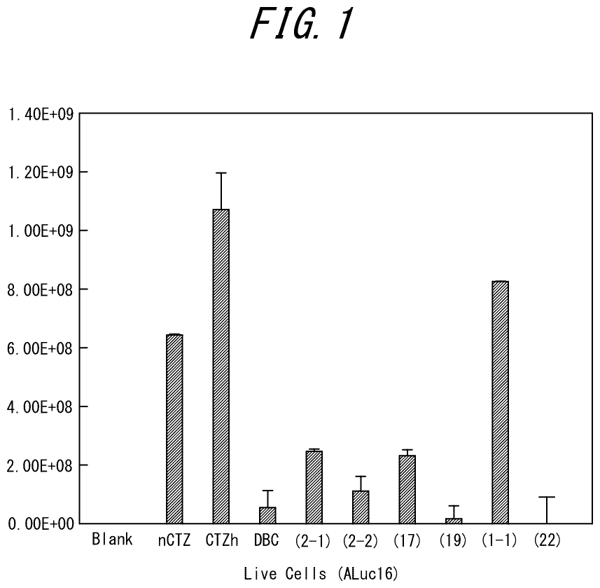

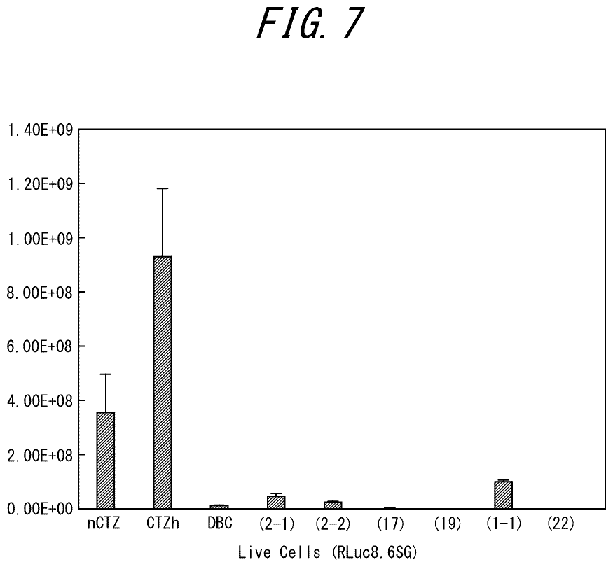

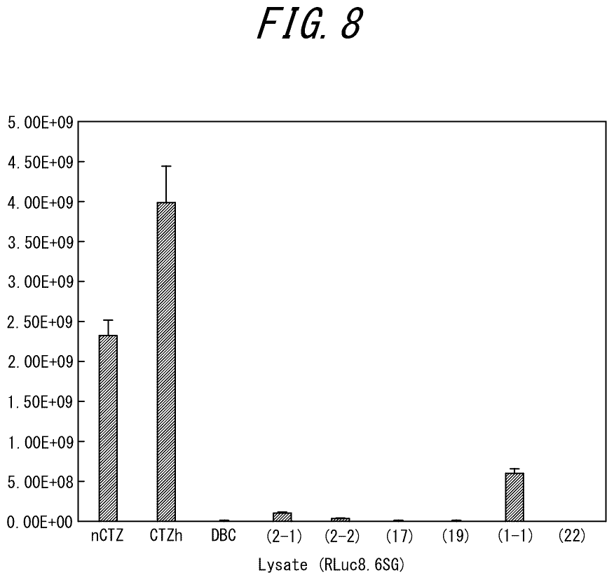

The results are shown in FIGS. 1, 2, 4, 5, 7, 8, 10, and 11. In FIGS. 1, 2, 4, 5, 7, 8, 10, and 11, the compound represented by structural formula (1-1) is indicated as “(1-1)”, the compound represented by structural formula (2-1) is indicated as “(2-1)”, the compound represented by structural formula (2-2) is indicated as “(2-2)”, the comparative compound (17) is indicated as “(17)”, the comparative compound (19) is indicated as “(19)”, and the comparative compound (22) is indicated as “(22)”.

### Evaluation of Bioluminescence Spectrum

The COS-7 cells were seeded in a 6-well microplate and were cultured in a CO₂ incubator until 70% of the bottom area was covered by the cells. Using TransIT-LT1 reagent (Mirus), the plasmid encoding each of the following marine bioluminescent enzymes was transiently expressed in cells in each well.

(i) ALuc16

(ii) ALuc47

(iii) Renilla luciferase 8.6-535SG (RLuc8.6SG)

(iv) NanoLuc

After this lipofection, the cells were cultured in a CO₂ incubator for one day. After the cell medium was completely removed from the 6-well microplate, 200 μL of cell lysis reagent (lysate) available from Promega K. K. was added to each well and the microplate was incubated at room temperature for 15 minutes.

Twenty microliters of this lysate were dispensed into each PCR tube, and 20 μL of the 100 μM diluted luminescent substrate solution was added at the time of luminescence brightness evaluation. The PCR tubes were immediately placed in a spectrophotometer (AB-1850, ATTO Corporation), and the bioluminescence spectrum was measured in the high-sensitivity mode with integration modes of 0.5 seconds, 5 seconds, 10 seconds, and 30 seconds.

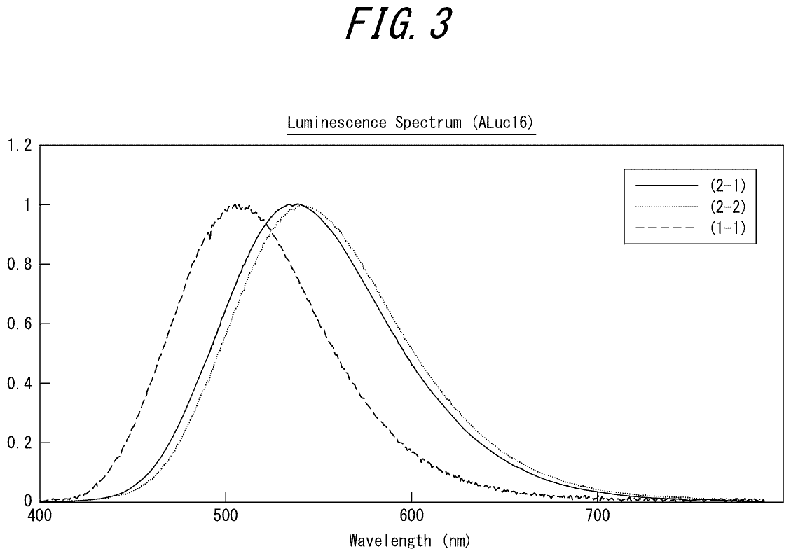

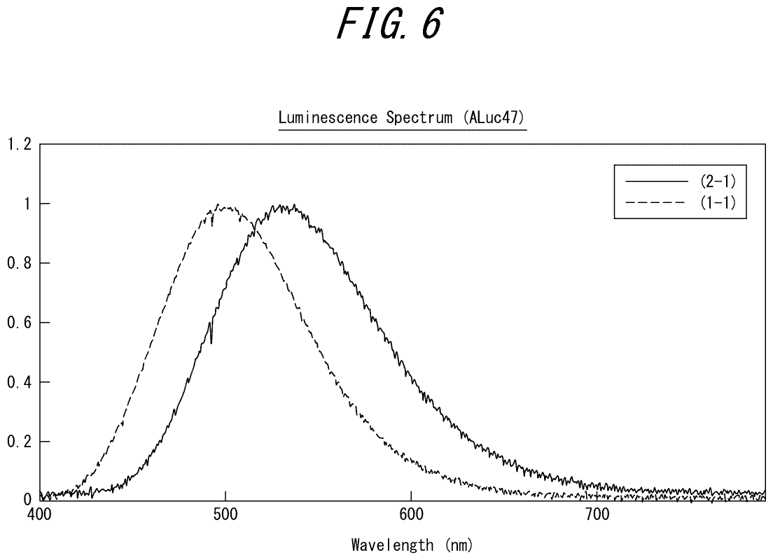

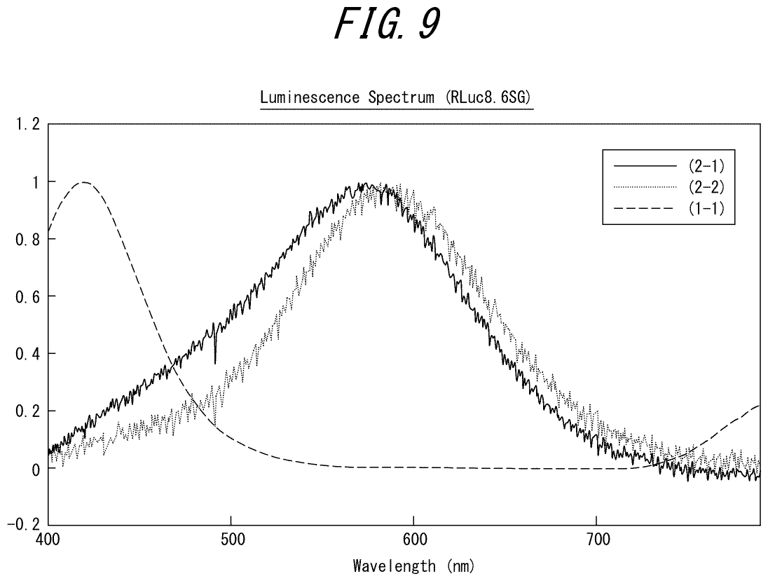

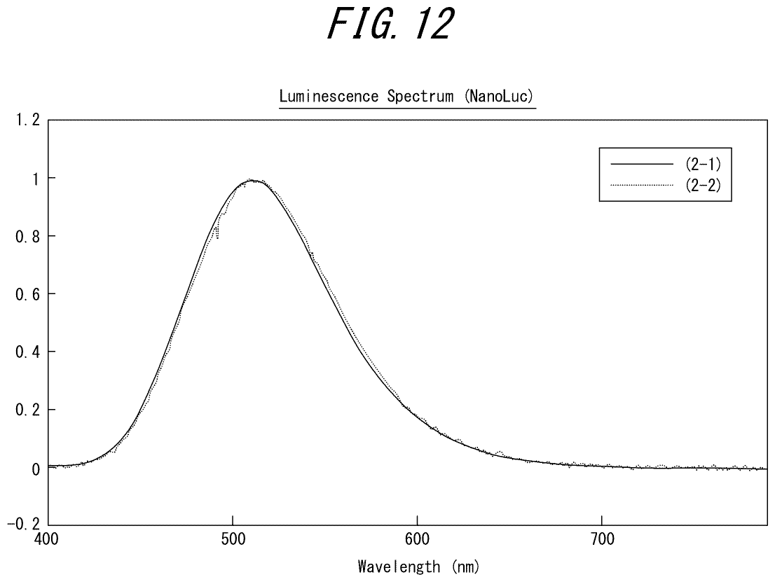

The results are shown in FIGS. 3, 6, 9, and 12. In FIGS. 3, 6, 9, and 12, the compound represented by structural formula (1-1) is indicated as “(1-1)”, the compound represented by structural formula (2-1) is indicated as “(2-1)”, and the compound represented by structural formula (2-2) is indicated as “(2-2)”.

### Results

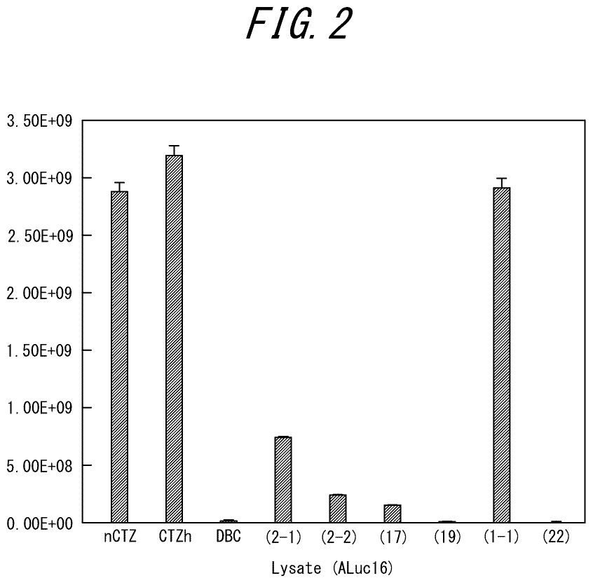

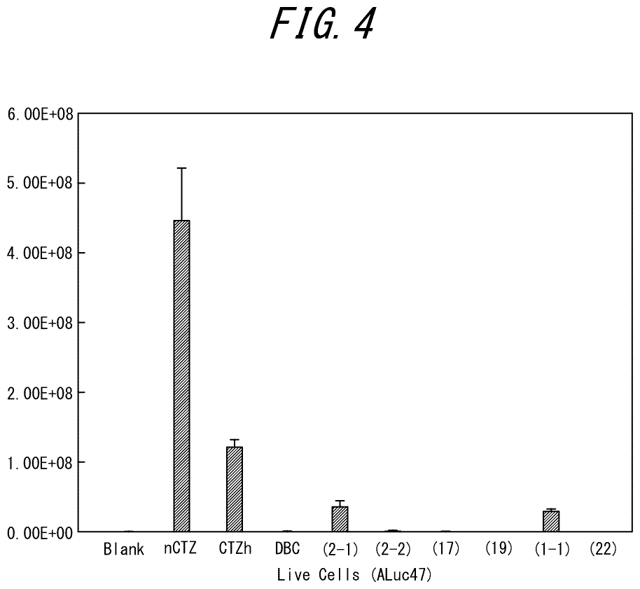

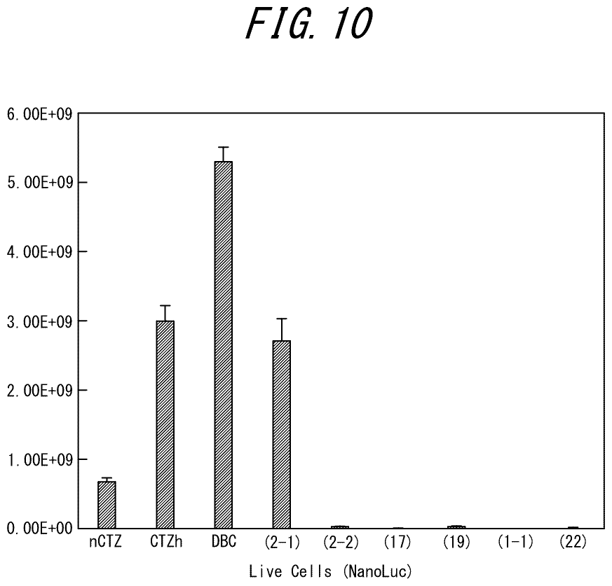

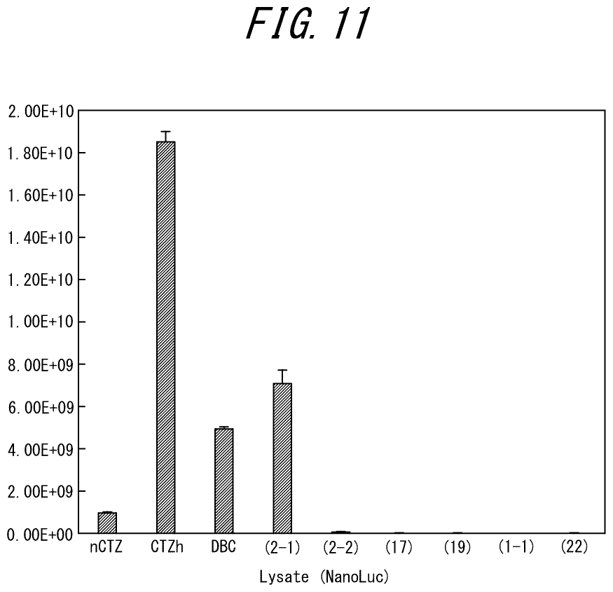

As shown in FIGS. 1 and 2, the compound represented by structural formula (1-1) showed high luminescence at high brightness for ALuc16. Furthermore, as shown in FIGS. 4, 5, 7, and 8, the compound represented by structural formula (1-1) showed slight luminescence for ALuc47 and RLuc8.6SG. On the other hand, as shown in FIGS. 10 and 11, the compound represented by structural formula (1-1) showed no luminescence activity for NanoLuc.

Thus, it confirms that the compound represented by structural formula (1-1) has enzyme specificity for ALuc16.

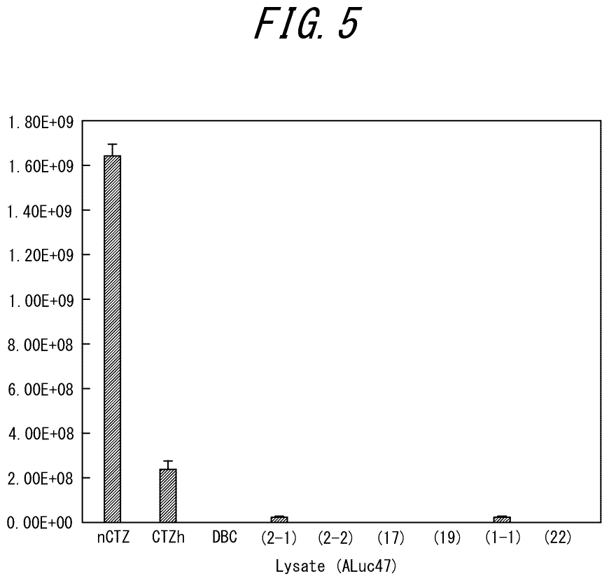

As shown in FIGS. 1, 2, 7, 8, 10, and 11, the compound represented by structural formula (2-1) showed luminescence activity for ALuc16, RLuc8.6SG, and NanoLuc. Furthermore, as shown in FIGS. 3, 9, and 12, the compound represented by structural formula (2-1) showed luminescence spectral peaks at 538 nm, 573 nm, and 507 nm for ALuc16, RLuc8.6SG, and NanoLuc, respectively. Furthermore, as shown in FIGS. 4 and 5, the compound represented by structural formula (2-1) also showed luminescence for ALuc47.

The compound represented by structural formula (2-2) showed luminescence activity for ALuc16 and RLuc8.6SG as shown in FIGS. 1, 2, 7, and 8, and slight luminescence for ALuc47 and NanoLuc as shown in FIGS. 4, 10, and 11.

Thus, it confirms that the compounds represented by structural formula (2-1) or (2-2) also have enzyme specificity.

Furthermore, it confirms that the compounds represented by structural formulas (2-1) and (2-2) exhibited a wavelength shift of approximately 50 nm to the longer wavelength side, compared to natural coelenterazine and the like.

### INDUSTRIAL APPLICABILITY

The coelenterazine derivatives of the present disclosure can be used as luminescent substrates for marine bioluminescent enzymes. Additionally, they can be widely used in various bioassays due to their unique enzyme specificity, strong luminescence brightness, and luminescence properties on the longer wavelength side. For example, the luminescence properties shifted to the longer wavelength side enable easy visualization of molecular events occurring in deep tissues. Additionally, because they luminesce brighter compared to conventional substances, they can improve the detection sensitivity and detection limit of bioassays. Furthermore, the luminescence specificity of the luminescent substrate enables the specific detection of a particular specimen among many specimens. Such a multiplexing feature significantly improves the efficiency of bioassays and greatly contributes to reducing diagnostic costs.

1. A coelenterazine derivative represented by the following general formula (1): 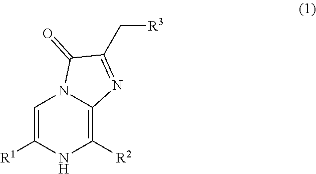
in the general formula (1), R¹ is represented by the following general formula (1-1-1), (1-1-2), (1-1-3), or (1-1-4): 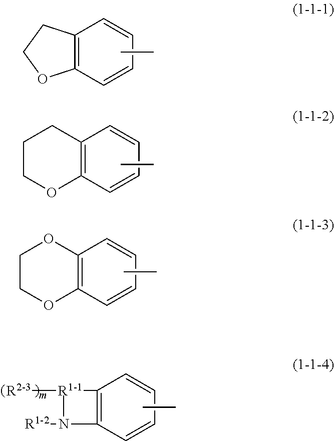
where R¹⁻¹ is a hydrocarbon group with 1 to 4 carbon atoms, each R¹⁻² is independently hydrogen or a hydrocarbon group with 1 to 3 carbon atoms, and m is an integer from 2 to 8,
R² is represented by —R^{2′} or —CH₂—R^{2′}, where R^{2′} is represented by the following general formula (1-2-1), (1-2-2), (1-2-3), (1-2-4), or (1-2-5): 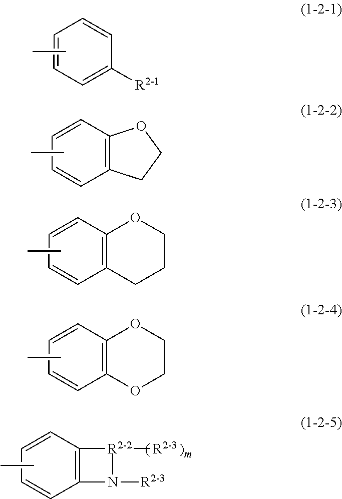
where R²⁻¹ is hydrogen, halogen, —N(R²⁻¹⁻¹)₂, or —OR²⁻¹⁻¹, where each R²⁻¹⁻¹ is independently hydrogen or a hydrocarbon group with 1 to 3 carbon atoms, R²⁻² is a hydrocarbon group with 1 to 4 carbon atoms, each R²⁻³ is independently hydrogen or a hydrocarbon group with 1 to 3 carbon atoms, and m is an integer from 2 to 8, and
R³ is represented by the following general formula (1-3-1), (1-3-2), or (1-3-3): 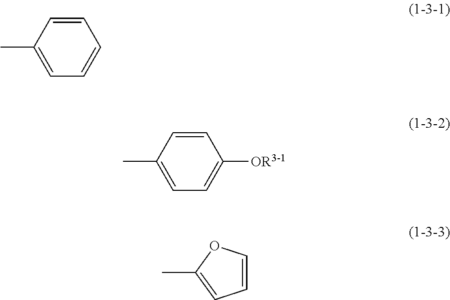
where R³⁻¹ is hydrogen or a hydrocarbon group with 1 to 3 carbon atoms; or the following general formula (2): 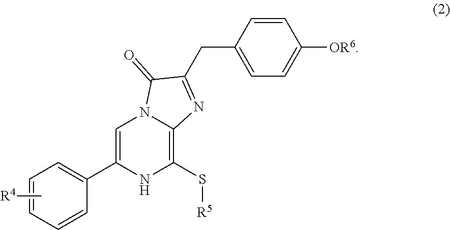
in the general formula (2), R⁴ is hydrogen, —(CH₂)ₙ—OR⁴⁻¹, —N(R⁴⁻¹)₂, or —CF₃, where each R⁴⁻¹ is independently hydrogen or a hydrocarbon group with 1 to 3 carbon atoms, and n is an integer from 0 to 3,
R⁵ is represented by the following general formula (2-5-1), (2-5-2), (2-5-3), (2-5-4), or (2-5-5): 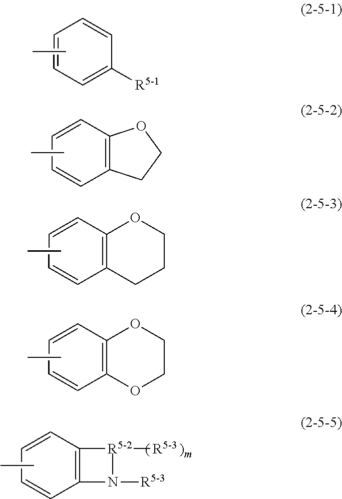
where R⁵⁻¹ is hydrogen, halogen, —N(R⁵⁻¹⁻¹)₂, or —OH, where each R⁵⁻¹⁻¹ is independently hydrogen or a hydrocarbon group with 1 to 3 carbon atoms, R^{5 2} is a hydrocarbon group with 1 to 4 carbon atoms, each R⁵⁻³ is independently hydrogen or a hydrocarbon group with 1 to 3 carbon atoms, and m is an integer from 2 to 8, and
R⁶ is hydrogen or a hydrocarbon group with 1 to 3 carbon atoms.

2. The coelenterazine derivative according to claim 1, which is represented by the above general formula (1), where R¹ is represented by the above general formula (1-1-3).

3. The coelenterazine derivative according to claim 1, which is represented by the above general formula (1), where R² is represented by —CH₂—R^{2′}, where R^{2′} is represented by the above general formula (1-2-1).

4. The coelenterazine derivative according to claim 1, which is represented by the above general formula (1), where R³ is represented by the above general formula (1-3-2).

5. The coelenterazine derivative according to claim 1, which is represented by the following structural formula (1-1):

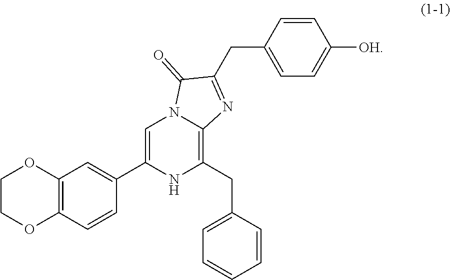 [SMILES: O=c1c(Cc2ccc(O)cc2)nc2c(Cc3ccccc3)[nH]c(-c3ccc4c(c3)OCCO4)cn1-2]

6. The coelenterazine derivative according to claim 1, which is represented by the above general formula (2), where R⁴ is represented by —(CH₂)ₙ—OR⁴⁻¹.

7. The coelenterazine derivative according to claim 1, which is represented by the above general formula (2), where R⁵ is represented by the above general formula (2-5-1).

8. The coelenterazine derivative according to claim 1, which is represented by the above general formula (2), where R⁶ is hydrogen.

9. The coelenterazine derivative according to claim 1, which is represented by the following structural formula (2-1) or (2-2):

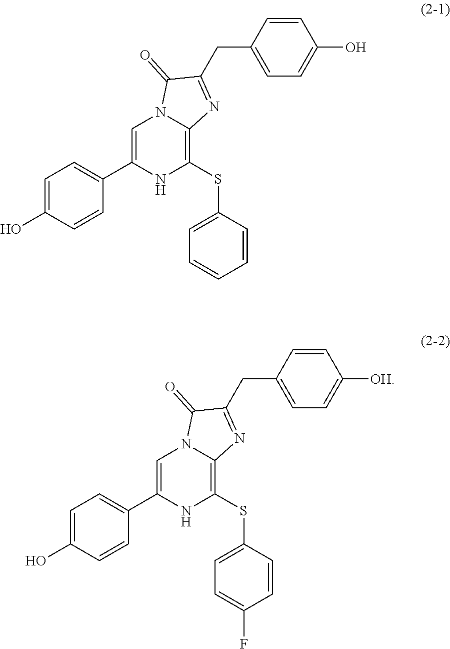 [SMILES: O=c1c(Cc2ccc(O)cc2)nc2c(Sc3ccccc3)[nH]c(-c3ccc(O)cc3)cn1-2 ; O=c1c(Cc2ccc(O)cc2)nc2c(Sc3ccc(F)cc3)[nH]c(-c3ccc(O)cc3)cn1-2]

10. The coelenterazine derivative according to claim 2, which is represented by the above general formula (1), where R² is represented by —CH₂—R^{2′}, where R^{2′} is represented by the above general formula (1-2-1).

11. The coelenterazine derivative according to claim 2, which is represented by the above general formula (1), where R³ is represented by the above general formula (1-3-2).

12. The coelenterazine derivative according to claim 3, which is represented by the above general formula (1), where R³ is represented by the above general formula (1-3-2).

13. The coelenterazine derivative according to claim 6, which is represented by the above general formula (2), where R⁵ is represented by the above general formula (2-5-1).

14. The coelenterazine derivative according to claim 6, which is represented by the above general formula (2), where R⁶ is hydrogen.

15. The coelenterazine derivative according to claim 7, which is represented by the above general formula (2), where R⁶ is hydrogen.

16. The coelenterazine derivative according to claim 10, which is represented by the above general formula (1), where R³ is represented by the above general formula (1-3-2).

17. The coelenterazine derivative according to claim 13, which is represented by the above general formula (2), where R⁶ is hydrogen.
